(19) United States
(12) Patent Application Publication
(10) Pub. No.: US 2017/0198266 A1
(43) Pub. Date: Jul. 13, 2017

(54) CONSTRUCTS AND METHODS FOR PLANT TRANSFORMATION

(71) Applicant: PIONEER HI-BRED INTERNATIONAL, INC., Johnston, IA (US)

(72) Inventors: Ajith Anand, West Des Moines, IA (US); Myeong-Je Cho, Sunnyvale, CA (US); William Gordon-Kamm, Urbandale, IA (US)

(73) Assignee: Pioneer HI Breed Internaional Inc., Johnston, IA (US)

(21) Appl. No.: 15/316,625

(22) PCT Filed: Jun. 5, 2015
(86) PCT No.: PCT/US15/34343

§ 371 Date: Dec. 6, 2016

Related U.S. Application Data
(60) Provisional application No. 62/009,661, filed on Jun. 9, 2014.

(51) Int. Cl. C12N 9/12; C12N 15/82

(52) U.S. Cl. CPC C12N 9/1241; C12N 15/8216; C12N 15/8286; C12N 15/8273; C12N 15/8209; C12Y 207/07

(57) ABSTRACT

Compositions and transformation methods to increase the frequency of plants in a population of transformed plants which have a single copy of the target polynucleotide of interest are provided. The frequency of plants in a population of transformed plants containing no contaminating vector backbone sequence may be increased. The methods and compositions provide for a greater number of transgenic events having single copy inserts and no contaminating vector backbone sequence.

### FIELD OF THE INVENTION

The present invention relates to the field of plant molecular biology, specifically increasing the number or ratio of single plant transformation events.

### BACKGROUND

Cultivated crops for food and fiber have substantial commercial value throughout the world. The development of scientific methods useful in improving the quantity and quality of agricultural crops is therefore of commercial interest. Significant effort has been expended to improve the quality of cultivated crop species by conventional plant breeding. Methods of conventional plant breeding have been limited, however, to the movement of genes and traits between plant varieties.

In addition to traditional breeding techniques other desirable traits can be introduced by plant genetic engineering. Plant genetic engineering involves the transfer of a desired gene or genes of interest into the germline of plants. Such genes may be bred into or among the elite varieties of crop plants allowing the introduction of novel traits and the development of new classes of crop varieties which may exhibit improved disease resistance, herbicide tolerance, or increased nutritional value.

Agrobacterium has been widely used for the transformation of plants. Agrobacterium is a soil-borne phytopathogen that integrates a nucleic acid molecule (i.e., T-DNA) into the genome of a variety of receptive plant species. Agrobacterium-mediated transformation involves incubation of cells or tissues with the bacterium, followed by regeneration of plants from the transformed cells via a callus stage.

However, plant gene transfer results in independent transformants that show highly variable levels and patterns of expression. Thus, for the commercial development of a plant with a new trait, hundreds of independent transformants must be screened for the few with suitable transgene structure and expression. The percentage of useable transformation events remains inefficiently low.

### SUMMARY

DNA constructs, methods for increasing the single copy transformation ratio in a population of transformed plants, and transformed plants so produced are provided. The DNA constructs and methods include or utilize a sequence comprising a non-inducible promoter operably linked to (i) a nucleotide encoding a recombinase and (ii) a 3′ regulatory element, wherein at least the nucleotide encoding the recombinase is flanked by at least two recombinase target sites in parallel orientation. The construct includes a polynucleotide encoding a polypeptide or polyribonucleotide which can be upstream or downstream of this sequence and is operably linked to a second promoter operable in a plant cell. In certain embodiments, the non-inducible promoter, the 3′ regulatory element or a combination thereof of the sequence is also flanked by the at least two parallel orientation recombinase target sites. The recombinase target sites can be, for example, RS, gix, lox, FRT, rox, an integrase, an invertase, a resolvase, or a chimeric recombinase target sites.

The DNA constructs and methods can include a T-DNA construct. The DNA construct can further include a synthetic T-DNA transmission enhancer, which can be an overdrive sequence. The synthetic T-DNA transmission enhancer can be upstream of the right border sequence. The non-inducible promoter of the DNA constructs and methods can be, for example, a constitutive promoter, a tissue-specific promoter or an organ-specific promoter. The recombinase of the DNA constructs and methods can be, for example, one or more of a Cre recombinase, a FLP recombinase, an invertase, an integrase, a resolvase, a chimeric recombination, or any combination thereof.

The polynucleotide may encode a promoter hairpin, a microRNA or a non-coding RNA or a polypeptide. In certain embodiments, the polypeptide enhances insect resistance, drought tolerance and/or nitrogen use efficiency of the transgenic plants transformed with the DNA constructs. The polynucleotide, in certain embodiments, encodes a selectable marker and a second polypeptide.

Transgenic plants or plant parts comprising the DNA construct are provided, which can be a monocot or dicot. For example, the transgenic plant or plant part can be maize, sorghum, rice, wheat, sugarcane, oat, rye, triticale, millet, soybean, alfalfa, canola, cotton, or sunflower.

Methods for increasing the single copy transformation ratio in a population of transgenic plants are also provided. The methods result in a higher number of plants containing cells having a single copy of the polypeptide or polyribonucleotide of interest. The methods comprise introducing the DNA construct into a plurality of plant cells to produce a population of transgenic plants, wherein the recombinase is expressed in the plant cells and wherein the population of transgenic plants comprises an increased single copy transformation ratio.

The methods include the step of introducing the DNA constructs described herein into a plurality of plant cells to produce a population of transgenic plants, wherein the recombinase is expressed in the plant cells and wherein the population of transgenic plants comprise a higher number of plants containing cells having a single copy of the polypeptide or polyribonucleotide compared with control plants transformed with a control construct. The control construct may be, for example, a similar construct, but which does not contain the at least two parallel orientation recombinase target sites. The population of transgenic plants can reflect an increased number of plants which do not express a backbone of the DNA construct compared with the control plants.

### BRIEF DESCRIPTION OF THE FIGURES

FIG. 1 is a schematic representation of constructs using a single recombinase site.

FIG. 2 is a schematic representation of constructs using multiple recombinase sites.

FIG. 3 is a schematic representation of constructs having overdrive and CRE/loxP for event quality improvement.

FIG. 4 is a schematic representation showing maps of PHP353 and PHP350 containing DsRED/CRE gene cassettes for glyphosate selection after excision of LoxP cassette.

### DETAILED DESCRIPTION

High-frequency production of transformed plants having a single heterologous polynucleotide stably integrated into the genome is desirable in commercial crop product development. Transformed plants with a suitable transgene structure and expression pattern may have both a single copy of the transgene and the absence of contaminating backbone DNA from the insertion vector. For example, the constructs and methods may result in a population of plants which has an increased number of plants containing cells which do not contain a backbone of the construct which carried the transgene or which contain a single copy of the transgene, or both.

As used herein, the term “plant” includes whole plants, plant organs (e.g., leaves, stems, roots, etc.), seeds, plant cells, and progeny of same. Parts of transgenic plants are within the scope of the embodiments and comprise, for example, plant cells, protoplasts, tissues, callus, embryos, as well as, flowers, stems, fruits, leaves, and roots originating in transgenic plants or their progeny previously transformed with a DNA molecule of the embodiments and therefore consisting at least in part of transgenic cells.

As used herein, the term plant “part” or “parts” includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. The class of plants that can be used in the methods of the embodiments is generally as broad as the class of higher plants amenable to transformation techniques, including both monocotyledonous and dicotyledonous plants.

As used herein, the term “plant cell” includes, without limitation, protoplasts and cells of seeds, suspension cultures, embryos, meristematic regions, callus tissue, leaves, roots, shoots, gametophytes, sporophytes, pollen, and microspores.

Green tissue refers to those plant parts that, when grown under conditions that include a period of light contain chlorophyll and photosynthesize. Green tissue can include regenerative tissue, callus tissue, and in vitro-cultured tissue, such as containing multiple-shoot meristem-like structures. These tissues have a high percentage of cells capable of sustained cell division and are competent for regeneration over long periods.

### Constructs

Provided are DNA constructs which include one or more polynucleotides of interest for the production of single copy transformants in transgenic plants. The DNA constructs may be contained within a vector such as binary, ternary or T-DNA vectors. The DNA constructs can include a non-inducible promoter operably linked to a nucleotide encoding a recombinase and a 3′ regulatory element. At least two recombinase target sites flank either (i) the nucleotide encoding the recombinase, (ii) the promoter and the nucleotide encoding the recombinase, (iii) the 3′ regulatory element and the nucleotide encoding the recombinase, or (iv) the promoter, the nucleotide encoding the recombinase and the 3′ regulatory element. The DNA constructs also include a polynucleotide encoding a polypeptide or a polyribonucleotide operably linked to a second promoter operable in a plant cell, which polynucleotide may be upstream or downstream of the sequence encoding the non-inducible promoter, the recombinase and the 3′ regulatory element.

As used herein, “polynucleotide” includes reference to a deoxyribonucleotide polymer in either single- or double-stranded form. A polyribonucleotide includes reference to a ribonucleotide polymer in either single- or double-stranded form. The nucleotide constructs, nucleic acids, and nucleotide sequences of the embodiments encompass all complementary forms of such constructs, molecules, and sequences.

In some examples, the DNA construct further comprises one or more ancillary sequences. Ancillary sequences include linkers, adapters, regulatory regions, introns, restriction sites, enhancers, insulators, selectable markers, promoters, other sites that aid in vector construction or analysis, or any combination thereof. The DNA construct may include one or more of a polynucleotide encoding a polypeptide or polyribonucleotide of interest, a promoter, selectable marker, recombinase coding sequence, recombination sites, a transmission enhancer, an ancillary sequence or any combination thereof and as set forth herein.

In some embodiments, an expression cassette may be used in the DNA construct. The expression cassette may include one or more of the components as set forth herein, which include, without limitation, a polynucleotide encoding a polypeptide or polyribonucleotide of interest, a promoter, such as a non-inducible promoter, a selectable marker, a recombinase coding sequence, two or more recombination sites, a transmission enhancer, an ancillary sequence or any combination thereof.

### Promoter of the Recombinase

A promoter is a region of DNA involved in recognition and binding of RNA polymerase and other proteins to initiate transcription. A plant promoter is a promoter capable of initiating transcription in a plant cell. For a review of plant promoters see Potenza et al. (2004) In Vitro Cell Dev Biol 40:1-22. The constructs include a non-inducible promoter which is functional in the transformed plant or tissue, such as callus or embryo, including immature embryo. The non-inducible promoter is operably linked to the recombinase coding sequence and directs expression of the recombinase coding sequence.

As used herein, “a non-inducible promoter” is a promoter that is expressed immediately upon transformation of a plant cell and promotes transcription in the plant cell of the recombinase in sufficient amounts for expression of a functional recombinase without the need for application of an exogenous signal. While certain promoters may drive expression differentially with varying environmental or developmental conditions, so long as the promoter drives expression of the recombinase in an amount sufficient to catalyze excision immediately upon transformation of the plant cell it is considered a non-inducible promoter of the recombinase. For example, certain promoters may be inducible by light, but would be considered non-inducible promoters as used herein, since transformation is performed under light conditions. Other tissue-specific promoters are considered non-inducible when they are transformed into the tissue in which they drive expression. For example, a callus specific promoter such as AXI is a non-inducible promoter when used to transform callus cells.

Suitable non-inducible promoters include constitutive promoters (such as those described herein for expression of the target polynucleotide), tissue-specific promoters, such as callus specific promoters for callus tissue, organ-specific promoters, and developmentally-regulated promoters. Examples of non-inducible promoters include, without limitation, cauliflower mosaic virus (CaMV) 35S, opine promoters, plant ubiquitin (Ubi), rice actin 1 (Act-1) and maize alcohol dehydrogenase 1 (Adh-1).

Inducible promoters not suitable for use include those that promote expression of a recombinase in sufficient amounts in a plant cell only when expressed in a tissue different from that being transformed, or following application of an exogenous signal which is incompatible or not present during the initial transformation process when the construct is introduced into the plant cell, or a combination thereof. The exogenous signal can be a chemical contacted with the plant cell, a change in the environment, such as a stress, heat, water, salinity, or biotic factor such as pathogen or insect attack.

In one example, at least one polynucleotide is under the control of an early embryo promoter. An early embryo is defined as the stages of embryo development including the zygote and the developing embryo up to the point where embryo maturation begins. An “early embryo promoter” is a promoter that drives expression predominately during the early stages of embryo development (i.e., before 15-18 DAP). Alternatively, the early embryo promoter can drive expression during both early and late stages. Early embryo promoters include, but are not limited to, to Lec 1 (WO 02/42424); cim1, a pollen and whole kernel specific promoter (WO 00/11177); the seed-preferred promoter end1 (WO 00/12733); and, the seed-preferred promoter end2 (WO 00/12733) and Ipt2 (U.S. Pat. No. 5,525,716). Additional promoters include smilps, an embryo specific promoter, and cz19B1a whole kernel specific promoter. See, for example, WO 00/11177, which is herein incorporated by reference. All of these references are herein incorporated by reference

Examples of inducible promoters, include, without limitation, heat shock promoters (such as HSP70 and HSP90), chemical inducible promoters such as the IN2 promoter, oxidative stress-inducible promoters, glutathione-inducible promoters, estradiol-inducible promoter, promoters that function in a glucocorticoid-inducible system, and promoters that function in an XVE inducible system.

### Promoter of the Target Polynucleotide

The sequence encoding a polyribonucleotide or polypeptide may also be under the control of a plant promoter. Such promoters may include, without limitation, constitutive, tissue-preferred, inducible or other promoters for expression in the host organism. Suitable constitutive promoters for use in a plant host cell include, for example, the core promoter of the Rsyn7 promoter and other constitutive promoters disclosed in WO1999/43838 and U.S. Pat. No. 6,072,050, the entire disclosures of which are herein incorporated by reference; the core CaMV 35S promoter; rice actin; ubiquitin; pEMU; MAS; ALS promoter (U.S. Pat. No. 5,659,026), the entire disclosure of which is herein incorporated by reference and the like. Other constitutive promoters include, for example, those discussed in U.S. Pat. Nos. 5,608,149; 5,608,144; 5,604,121; 5,569,597; 5,466,785; 5,399,680; 5,268,463; 5,608,142 and 6,177,611, the entire disclosures of which are herein incorporated by reference.

Depending on the desired outcome, it may be beneficial to control expression of the polyribonucleotide or polypeptide with an inducible promoter. Wound-inducible promoters, which may respond to damage caused by insect feeding include the potato proteinase inhibitor (pin II) gene promoter; wun1 and wun2 disclosed in U.S. Pat. No. 5,428,148, the entire disclosure of which is herein incorporated by reference; systemin; WIP1; MPI gene promoter and the like.

Additionally, pathogen-inducible promoters may be employed in the methods and nucleotide constructs of the embodiments. Such pathogen-inducible promoters include those from pathogenesis-related proteins (PR proteins), which are induced following infection by a pathogen; e.g., PR proteins, SAR proteins, beta-1,3-glucanase, chitinase, etc. See, for example, WO1999/43819, the entire disclosure of which is herein incorporated by reference.

Of interest are promoters that are expressed locally at or near the site of pathogen infection. See, for example, U.S. Pat. No. 5,750,386 (nematode-inducible) the entire disclosure of which is herein incorporated by reference and the references cited therein. Of particular interest is the inducible promoter for the maize PRms gene, whose expression is induced by the pathogen Fusarium moniliforme.

Chemical-regulated promoters can be used to modulate the expression of a gene in a plant through the application of an exogenous chemical regulator. Depending upon the objective, the promoter may be a chemical-inducible promoter, where application of the chemical induces gene expression or a chemical-repressible promoter, where application of the chemical represses gene expression. Chemical-inducible promoters are known in the art and include, but are not limited to, the maize In2-2 promoter, which is activated by benzenesulfonamide herbicide safeners, the maize GST promoter, which is activated by hydrophobic electrophilic compounds that are used as pre-emergent herbicides, and the tobacco PR-1α promoter, which is activated by salicylic acid. Other chemical-regulated promoters of interest include steroid-responsive promoters such as the glucocorticoid-inducible promoter and the tetracycline-inducible and tetracycline-repressible promoters (see, for example, U.S. Pat. Nos. 5,814,618 and 5,789,156, the entire disclosures of which are herein incorporated by reference.

Tissue-preferred promoters can be utilized to target enhanced polypeptide expression within a particular plant tissue. Tissue-preferred promoters are known in the art and include those promoters which can be modified for weak expression.

Leaf-preferred, root-preferred or root-specific promoters can be selected from those known in the art, or isolated de novo from various compatible species. Examples of root-specific promoter include those promoters of the soybean glutamine synthetase gene, the control element in the GRP 1.8 gene of French bean, the mannopine synthase (MAS) gene of Agrobacterium tumefaciens, and the full-length cDNA clone encoding cytosolic glutamine synthetase (GS). Root-specific promoters also include those isolated from hemoglobin genes from the nitrogen-fixing nonlegume Parasponia andersonii and the related non-nitrogen-fixing nonlegume Trema tomentosa, promoters of the highly expressed roIC and roID root-inducing genes of Agrobacterium rhizogenes, the root-tip specific promoter of octopine synthase, and the root-specific promoter of the TR2′ and TR1′genes, which are also stimulated by wounding in leaf tissue, an especially desirable combination of characteristics for use with an insecticidal or larvicidal gene. Additional root-preferred promoters include the VfENOD-GRP3 gene promoter and rolB promoter. See, e.g., U.S. Pat. Nos. 5,837,876; 5,750,386; 5,633,363; 5,459,252; 5,401,836; 5,110,732 and 5,023,179, the entire disclosures of which are herein incorporated by reference. Arabidopsis thaliana root-preferred regulatory sequences are disclosed in US20130117883, the entire disclosure of which is herein incorporated by reference.

“Seed-preferred” promoters include both “seed-specific” promoters (those promoters active during seed development such as promoters of seed storage proteins) as well as “seed-germinating” promoters (those promoters active during seed germination). Such seed-preferred promoters include, but are not limited to, Cim1 (cytokinin-induced message); cZ19B1 (maize 19 kDa zein); and milps (myo-inositol-1-phosphate synthase) (see, U.S. Pat. No. 6,225,529, the entire disclosure of which is herein incorporated by reference). Gamma-zein and GIb-1 are endosperm-specific promoters. For dicots, seed-specific promoters include, but are not limited to, Kunitz trypsin inhibitor 3 (KTi3), bean β-phaseolin, napin, β-conglycinin, glycinin 1, soybean lectin, cruciferin, and the like. For monocots, seed-specific promoters include, but are not limited to, maize 15 kDa zein, 22 kDa zein, 27 kDa zein, g-zein, waxy, shrunken 1, shrunken 2, globulin 1, etc. See also, WO2000/12733, where seed-preferred promoters from end1 and end2 genes are disclosed, the entire disclosure of which is herein incorporated by reference. In dicots, seed specific promoters include, but are not limited to, the seed coat promoter from Arabidopsis, pBAN; and the early seed promoters from Arabidopsis, p26, p63, and p63tr (U.S. Pat. Nos. 7,294,760 and 7,847,153, the entire disclosures of which are herein incorporated by reference). A promoter that has “preferred” expression in a particular tissue is expressed in that tissue to a greater degree than in at least one other plant tissue. Some tissue-preferred promoters show expression almost exclusively in the particular tissue.

The above list of promoters is not meant to be limiting. Any appropriate promoter can be used in the embodiments.

### Selectable Marker

The DNA construct may, or may not include a sequence encoding a selectable marker. In some embodiments, the selectable marker gene facilitates the selection of transformed cells or tissues. Selectable marker sequences include sequences encoding antibiotic resistance, such as neomycin phosphotransferase II (NEO) and hygromycin phosphotransferase (HPT), as well as sequences conferring resistance to herbicidal compounds, such as glufosinate ammonium, bromoxynil, imidazolinones, and 2,4-dichlorophenoxyacetate (2,4-D). Additional examples of suitable selectable marker sequences include, but are not limited to, sequences encoding resistance to chloramphenicol, methotrexate, streptomycin, spectinomycin, bleomycin, sulfonamide, bromoxynil, phosphinothricin, and glyphosate (see for example US Patent Publication Nos. 20030083480 and 20040082770, the entire disclosures of which are herein incorporated by reference).

The above list of selectable marker sequences is not meant to be limiting. Any selectable marker coding sequence can be used in the embodiments.

### Recombinase Sequences and Recombination Sites

The DNA construct includes a sequence encoding a recombinase and its corresponding recombination sites. The recombinase is flanked by the two or more recombination sites and the recombination sites are in the same parallel orientation. Parallel orientation means that the two or more recombination sequences are either both or all in the 3′ to 5′ orientation, or are both or all in the 5′ to 3′ orientation. A set of recombination sites arranged in the same orientation, as described herein, will result in excision, rather than inversion, of the intervening DNA sequence between the recombination sites. Inversion occurs when the recombination sites are oriented in opposite or mixed orientations.

A recombinase, also referred to as a site-specific recombinase, is a polypeptide that catalyzes conservative site-specific recombination between its compatible recombination sites. A recombinase can include native polypeptides, variants and/or fragments that retain recombinase activity. A sequence encoding a recombinase can include native polynucleotides, variants and/or fragments that encode a recombinase that retains recombinase activity. Suitable recombinases that are encoded include native recombinases or biologically active fragments or variants of the recombinase, such as those which catalyze conservative site-specific recombination between specified recombination sites. A native polypeptide or polynucleotide comprises a naturally occurring amino acid sequence or nucleotide sequence. The recombinase and its compatible sites may be referred to as a recombinase system. Any recombinase system can be used. In some embodiments recombinases from the integrase and resolvase families are used.

In some embodiments, a chimeric recombinase can be used. A chimeric recombinase is a recombinant fusion protein which is capable of catalyzing site-specific recombination between recombination sites that originate from different recombination systems. For example, if the set of recombination sites comprises a FRT site and a LoxP site, a chimeric FLP/Cre recombinase or active variant or fragment thereof can be used, or both recombinases may be separately provided. Methods for the production and use of such chimeric recombinases or active variants or fragments thereof are described, for example, in WO99/25840, the entire disclosure of which is herein incorporated by reference.

Any suitable recombination site or set of recombination sites may be utilized in the methods and compositions, including, but not limited to: a FRT site, a functional variant of a FRT site, a LOX site, and functional variant of a LOX site, any combination thereof, or any other combination of recombination sites known. Recombinase systems which may be used include, without limitation, the Gin recombinase of phage Mu, the Pin recombinase of E. coli, the PinB, PinD and PinF from Shigella, and the R/RS system of Zygosaccharomyces rouxii.

Functional variants include chimeric recombination sites, such as an FRT site fused to a LOX site. For example, recombination sites from the Cre/Lox site-specific recombination system can be used. Such recombination sites include, for example, native LOX sites and various functional variants of LOX (see, e.g., U.S. Pat. No. 6,465,254 and WO01/111058, the entire disclosures of which are herein incorporated by reference). Recombinogenic modified FRT recombination sites can be used in various in vitro and in vivo site-specific recombination methods that allow for the targeted integration, exchange, modification, alteration, excision, inversion, and/or expression of a nucleotide sequence of interest, see for example, WO99/25821, WO99/25854, WO99/25840, WO99/25855, WO99/25853, and WO2007/011733, the entire disclosures of which are herein incorporated by reference.

Suitable recombinase (includes integrase) sites are shown in Table 1:

TABLE 1
Recombinase Sites

| | |
|---|---|
| Cinh | RS2 (113 bp) |
| ParA | MRS (133 bp) |
| Tn1721 | res (120 bp) |
| Tn5053 | res (176 bp) |
| PhiC31 | attP (40 bp) & attB (34 bp) |
| TP901-1 | attP (56 bp) & attB (43 bp) |
| Bxb1 | attP (39 bp) & attB (34 bp) |
| U153 | attP (57 bp) & attB (51 bp) |

### Transmission Enhancer

In some embodiments, the DNA construct may contain a synthetic T-DNA transmission enhancer. Examples of such enhancers include the Overdrive (OD) sequence and T-DNA transfer stimulator sequence (TSS). The T-DNA constructs include a left border sequence and a right border sequence and the synthetic T-DNA transmission enhancer can be upstream of the right border sequence.

### Cell Proliferation Factors

Any of a number of cell proliferation factors can be used in the constructs and methods of the embodiments. In some embodiments, a cell proliferation factor in the AP2/ERF family of proteins can be used. The AP2/ERF family of proteins is a plant-specific class of putative transcription factors that regulate a wide-variety of developmental processes and are characterized by the presence of an AP2/ERF DNA binding domain. The AP2/ERF proteins have been subdivided into distinct subfamilies based on the presence of conserved domains. Initially, the family was divided into two subfamilies based on the number of DNA binding domains, with the ERF subfamily having one DNA binding domain, and the AP2 subfamily having two DNA binding domains. As more sequences were identified, the family was subsequently subdivided into five subfamilies: AP2, DREB, ERF, RAV, and others. Members of the APETALA2 (AP2) family of proteins function in a variety of biological events including, but not limited to, development, plant regeneration, cell division, embryogenesis, cell proliferation. The AP2 family includes but is not limited to: AP2, ANT, Glossy15, AtBBM, BnBBM, and ODP2 from maize.

### Coding Sequences for Polypeptides and Polyribonucleotides of Interest

The DNA constructs include a promoter that has a polynucleotide encoding a polyribonucleotide or a polypeptide operably linked to it. The promoter may be the same, similar or different from the non-inducible promoter that is operably linked to the nucleotide encoding a recombinase.

In certain embodiments the nucleic acid sequences of the embodiments can be stacked with any combination of polynucleotide sequences of interest in order to create plants with a desired phenotype.

Suitable polynucleotides include those encoding Bacillus thuringiensis delta-endotoxins, see for example U.S. Pat. Nos. 5,188,960; 5,689,052; 5,880,275; 5,986,177; 6,023,013, 6,060,594, 6,063,597, 6,077,824, 6,620,988, 6,642,030, 6,713,259, 6,893,826, 7,105,332; 7,179,965, 7,208,474; 7,227,056, 7,288,643, 7,323,556, 7,329,736, 7,449,552, 7,468,278, 7,510,878, 7,521,235, 7,544,862, 7,605,304, 7,696,412, 7,629,504, 7,705,216, 7,772,465, 7,790,846, 7,858,849 and WO1991/14778; WO1999/31248; WO2001/12731; WO1999/24581 and WO1997/40162 the entire disclosures of which are herein incorporated by reference. Examples of delta-endotoxins also include but are not limited to Cry1A proteins of U.S. Pat. Nos. 5,880,275 and 7,858,849 the entire disclosures of which are herein incorporated by reference; a DIG-3 or DIG-11 toxin (N-terminal deletion of α-helix 1 and/or α-helix 2 variants of Cry proteins such as CrylA) of U.S. Pat. No. 8,304,604 and 8.304,605 the entire disclosures of which are herein incorporated by reference; Cry1B of US20060112447 the entire disclosure of which is herein incorporated by reference; Cry1C of U.S. Pat. No. 6,033,874 the entire disclosure of which is herein incorporated by reference; Cry1F of U.S. Pat. Nos. 5,188,960, 6,218,188 the entire disclosures of which are herein incorporated by reference; Cry1A/F chimeras of U.S. Pat. Nos. 7,070,982; 6,962,705 and 6,713,063 the entire disclosures of which are herein incorporated by reference; a Cry2 protein such as Cry2Ab protein of U.S. Pat. No. 7,064,249 the entire disclosure of which is herein incorporated by reference; a Cry3A protein including but not limited to an engineered hybrid insecticidal protein (eHIP) created by fusing unique combinations of variable regions and conserved blocks of at least two different Cry proteins (US2010/0017914 the entire disclosure of which is herein incorporated by reference); a Cry4 protein; a Cry5 protein; a Cry6 protein; Cry8 proteins of U.S. Pat. Nos. 7,329,736, 7,449,552, 7,803,943, 7,476,781, 7,105,332, 7,378,499 and 7,462,760 the entire disclosures of which are herein incorporated by reference; a Cry34Ab1 protein of U.S. Pat. Nos. 6,127,180, 6,624,145 and 6,340,593 the entire disclosures of which are herein incorporated by reference; a CryET33 and CryET34 protein of U.S. Pat. Nos. 6,248,535, 6,326,351, 6,399,330, 6,949,626, 7,385,107 and 7,504,229 the entire disclosures of which are herein incorporated by reference; a CryET33 and CryET34 homologs of US2006/0191034, 2012/0278954, and WO2012/139004 the entire disclosures of which are herein incorporated by reference; a Cry35Ab1 protein of U.S. Pat. Nos. 6,083,499, 6,548,291 and 6,340,593 the entire disclosures of which are herein incorporated by reference; TIC807 of US 2008/0295207 the entire disclosure of which is herein incorporated by reference; ET29, ET37, TIC809, TIC810, TIC812, TIC127, TIC128 of PCT/US2006/033867 the entire disclosure of which is herein incorporated by reference; AXMI-027, AXMI-036, and AXMI-038 of U.S. Pat. No. 8,236,757 the entire disclosure of which is herein incorporated by reference; AXMI-031, AXMI-039, AXMI-040, AXMI-049 of U.S. Pat. No. 7,923,602 the entire disclosure of which is herein incorporated by reference; AXMI-018, AXMI-020, and AXMI-021 of WO2006/083891 the entire disclosure of which is herein incorporated by reference; AXMI-010 of WO2005/038032 the entire disclosure of which is herein incorporated by reference; AXMI-003 of WO2005/021585 the entire disclosure of which is herein incorporated by reference; AXMI-008 of US 2004/0250311 the entire disclosure of which is herein incorporated by reference; AXMI-006 of US 2004/0216186 the entire disclosure of which is herein incorporated by reference; AXMI-007 of US 2004/0210965 the entire disclosure of which is herein incorporated by reference; AXMI-009 of US 2004/0210964 the entire disclosure of which is herein incorporated by reference; AXMI-014 of US 2004/0197917 the entire disclosure of which is herein incorporated by reference; AXMI-004 of US 2004/0197916 the entire disclosure of which is herein incorporated by reference; AXMI-028 and AXMI-029 of WO2006/119457 the entire disclosure of which is herein incorporated by reference; AXMI-007, AXMI-008, AXMI-0080rf2, AXMI-009, AXMI-014 and AXMI-004 of WO2004/074462 the entire disclosure of which is herein incorporated by reference; AXMI-150 of U.S. Pat. No. 8,084,416 the entire disclosure of which is herein incorporated by reference; AXMI-205 of US20110023184 the entire disclosure of which is herein incorporated by reference; AXMI-011, AXMI-012, AXMI-013, AXMI-015, AXMI-019, AXMI-044, AXMI-037, AXMI-043, AXMI-033, AXMI-034, AXMI-022, AXMI-023, AXMI-041, AXMI-063, and AXMI-064 of US 2011/0263488 the entire disclosure of which is herein incorporated by reference; AXMI-R1 and related proteins of US 2010/0197592 the entire disclosure of which is herein incorporated by reference; AXMI221Z, AXMI222z, AXMI223z, AXMI224z and AXMI225z of WO2011/103248 the entire disclosure of which is herein incorporated by reference; AXMI218, AXMI219, AXMI220, AXMI226, AXMI227, AXMI228, AXMI229, AXMI230, and AXMI231 of WO11/103247 the entire disclosure of which is herein incorporated by reference; AXMI-115, AXMI-113, AXMI-005, AXMI-163 and AXMI-184 of U.S. Pat. No. 8,334,431 the entire disclosure of which is herein incorporated by reference; AXMI-001, AXMI-002, AXMI-030, AXMI-035, and AXMI-045 of US 2010/0298211; AXMI-066 and AXMI-076 of US20090144852 the entire disclosure of which is herein incorporated by reference; AXMI128, AXMI130, AXMI131, AXMI133, AXMI140, AXMI141, AXMI142, AXMI143, AXMI144, AXMI146, AXMI148, AXMI149, AXMI152, AXMI153, AXMI154, AXMI155, AXMI156, AXMI157, AXMI158, AXMI162, AXMI165, AXMI166, AXMI167, AXMI168, AXMI169, AXMI170, AXMI171, AXMI172, AXMI173, AXMI174, AXMI175, AXMI176, AXMI177, AXMI178, AXMI179, AXMI180, AXMI181, AXMI182, AXMI185, AXMI186, AXMI187, AXMI188, AXMI189 of U.S. Pat. No. 8,318,900 the entire disclosure of which is herein incorporated by reference; AXMI079, AXMI080, AXMI081, AXMI082, AXMI091, AXMI092, AXMI096, AXMI097, AXMI098, AXMI099, AXMI100, AXMI101, AXMI102, AXMI103, AXMI104, AXMI107, AXMI108, AXMI109, AXMI110, AXMI111, AXMI112, AXMI114, AXMI116, AXMI117, AXMI118, AXMI119, AXMI120, AXMI121, AXMI122, AXMI123, AXMI124, AXMI1257, AXMI1268, AXMI127, AXMI129, AXMI164, AXMI151, AXMI161, AXMI183, AXMI132, AXMI138, AXMI137 of US 2010/0005543 the entire disclosure of which is herein incorporated by reference; and Cry proteins such as CrylA and Cry3A having modified proteolytic sites of U.S. Pat. No. 8,319,019 the entire disclosure of which is herein incorporated by reference; and a Cry1Ac, Cry2Aa and Cry1Ca toxin protein from Bacillus thuringiensis strain VBTS 2528 of US2011/0064710 the entire disclosure of which is herein incorporated by reference. Other Cry proteins are well known to one skilled in the art. More than one pesticidal proteins well known to one skilled in the art can also be expressed in plants such as Vip3Ab & Cry1Fa (US2012/0317682 the entire disclosure of which is herein incorporated by reference); Cry1BE & Cry1F (US2012/0311746 the entire disclosure of which is herein incorporated by reference); Cry1CA & Cry1AB (US2012/0311745 the entire disclosure of which is herein incorporated by reference); Cry1F & CryCa (US2012/0317681 the entire disclosure of which is herein incorporated by reference); Cry1DA & Cry1BE (US2012/0331590 the entire disclosure of which is herein incorporated by reference); Cry1DA & Cry1Fa (US2012/0331589 the entire disclosure of which is herein incorporated by reference); Cry1AB & Cry1BE (US2012/0324606 the entire disclosure of which is herein incorporated by reference); and Cry1Fa & Cry2Aa, Cry1l or Cry1E (US2012/0324605 the entire disclosure of which is herein incorporated by reference). Pesticidal proteins also include insecticidal lipases including lipid acyl hydrolases of U.S. Pat. No. 7,491,869 the entire disclosure of which is herein incorporated by reference. Pesticidal proteins also include VIP (vegetative insecticidal proteins) toxins of U.S. Pat. Nos. 5,877,012, 6,107,279, 6,137,033, 7,244,820, 7,615,686, and 8,237,020 the entire disclosures of which are herein incorporated by reference, and the like. Other VIP proteins are well known to one skilled in the art. Pesticidal proteins also include toxin complex (TC) proteins, obtainable from organisms such as Xenorhabdus, Photorhabdus and Paenibacillus (see, U.S. Pat. Nos. 7,491,698 and 8,084,418 the entire disclosures of which are herein incorporated by reference). Some TC proteins have “stand alone” insecticidal activity and other TC proteins enhance the activity of the stand-alone toxins produced by the same given organism. The toxicity of a “stand-alone” TC protein (from Photorhabdus, Xenorhabdus or Paenibacillus, for example) can be enhanced by one or more TC protein “potentiators” derived from a source organism of a different genus. There are three main types of TC proteins. As referred to herein, Class A proteins (“Protein A”) are stand-alone toxins. Class B proteins (“Protein B”) and Class C proteins (“Protein C”) enhance the toxicity of Class A proteins. Examples of Class A proteins are TcbA, TcdA, XptA1 and XptA2. Examples of Class B proteins are TcaC, TcdB, XptB1Xb and XptC1Wi. Examples of Class C proteins are TccC, XptC1Xb and XptB1Wi. Pesticidal proteins also include spider, snake and scorpion venom proteins. Examples of spider venom peptides include but are not limited to lycotoxin-1 peptides and mutants thereof (U.S. Pat. No. 8,334,366 the entire disclosure of which is herein incorporated by reference).

Other suitable polynucleotides include those encoding a hydrophobic moment peptide. See, WO1995/16776 and U.S. Pat. No. 5,580,852 the entire disclosures of which are herein incorporated by reference peptide derivatives of Tachyplesin which inhibit fungal plant pathogens) and PCT Application WO1995/18855 and U.S. Pat. No. 5,607,914 the entire disclosures of which are herein incorporated by reference (synthetic antimicrobial peptides that confer disease resistance).

Polynucleotides encoding antifungal proteins are also useful in the embodiments. See, e.g., U.S. Pat. Nos. 6,875,907, 7,498,413, 7,589,176, 7,598,346, 8,084,671, 6,891,085 and 7,306,946; the entire disclosures of which are herein incorporated by reference. Polynucleotides encoding LysM receptor-like kinases for the perception of chitin fragments as a first step in plant defense response against fungal pathogens (US 2012/0110696 the entire disclosures of which are herein incorporated by reference) are also useful in the embodiments.

Other suitable polynucleotides include those encoding detoxification peptides such as for fumonisin, beauvericin, moniliformin and zearalenone and their structurally related derivatives. For example, see, U.S. Pat. Nos. 5,716,820; 5,792,931; 5,798,255; 5,846,812; 6,083,736; 6,538,177; 6,388,171 and 6,812,380 the entire disclosures of which are herein incorporated by reference.

Various changes in phenotype are of interest including modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant. For example, down-regulation of stearoyl-ACP can increase stearic acid content of the plant. See, WO1999/64579 the entire disclosure of which is herein incorporated by reference; elevating oleic acid via FAD-2 gene modification and/or decreasing linolenic acid via FAD-3 gene modification (see, U.S. Pat. Nos. 6,063,947; 6,323,392; 6,372,965 and WO1993/11245 the entire disclosures of which are herein incorporated by reference); altering conjugated linolenic or linoleic acid content, such as in WO2001/12800 the entire disclosure of which is herein incorporated by reference; altering LEC1, AGP, Dek1, Superall, milps, various Ipa genes such as Ipa1, Ipa3, hpt or hggt. For example, see, WO2002/42424, WO1998/22604, WO2003/011015, WO2002/057439, WO2003/011015, U.S. Pat. Nos. 6,423,886, 6,197,561, 6,825,397, US2003/0079247, and US2003/0204870 the entire disclosures of which are herein incorporated by reference; polynucleotides encoding delta-8 desaturase for making long-chain polyunsaturated fatty acids (U.S. Pat. Nos. 8,058,571 and 8,338,152 the entire disclosures of which are herein incorporated by reference) and delta-9 desaturase for lowering saturated fats (U.S. Pat. No. 8,063,269 the entire disclosure of which is herein incorporated by reference); polynucleotides and encoded proteins associated with lipid and sugar metabolism regulation, in particular, lipid metabolism protein (LMP) used in methods of producing transgenic plants and modulating levels of seed storage compounds including lipids, fatty acids, starches or seed storage proteins and use in methods of modulating the seed size, seed number, seed weights, root length and leaf size of plants (EP 2404499 the entire disclosure of which is herein incorporated by reference); altering expression of a High-Level Expression of Sugar-Inducible 2 (HSI2) protein in the plant to increase or decrease expression of HSI2 in the plant. Increasing expression of HSI2 increases oil content while decreasing expression of HSI2 decreases abscisic acid sensitivity and/or increases drought resistance (US2012/0066794 the entire disclosure of which is herein incorporated by reference); expression of cytochrome b5 (Cb5) alone or with FAD2 to modulate oil content in plant seed, particularly to increase the levels of omega-3 fatty acids and improve the ratio of omega-6 to omega-3 fatty acids (US2011/0191904 the entire disclosure of which is herein incorporated by reference); polynucleotides encoding wrinkled1-like polypeptides for modulating sugar metabolism (U.S. Pat. No. 8,217,223 the entire disclosure of which is herein incorporated by reference).

Polynucleotides encoding polypeptides which alter phosphorus content are also useful in the embodiments. For example, by the introduction of a phytase-encoding gene that enhances breakdown of phytate, adding more free phosphate to the transformed plant; by reducing phytate content. In maize, this, for example, could be accomplished, by cloning and then re-introducing DNA associated with one or more of the alleles, such as the LPA alleles, identified in maize mutants characterized by low levels of phytic acid, such as in WO2005/113778 the entire disclosure of which is herein incorporated by reference and/or by altering inositol kinase activity as in WO2002/059324, US 2003/0009011, WO2003/027243, US2003/0079247, WO1999/05298, U.S. Pat. No. 6,197,561, U.S. Pat. No. 6,291,224, U.S. Pat. No. 6,391,348, WO2002/059324, US2003/0079247, WO1998/45448, WO1999/55882 and WO2001/04147 the entire disclosures of which are herein incorporated by reference; by altering thioredoxin such as NTR and/or TRX (see, U.S. Pat. No. 6,531,648. which is incorporated by reference in its entirety) and/or a gamma zein knock out or mutant such as cs27 or TUSC27 or en27 (see, U.S. Pat. No. 6,858,778 and US2005/0160488, US2005/0204418, the entire disclosures of which are herein incorporated by reference); nucleotide sequence of Streptococcus mutant fructosyltransferase gene; nucleotide sequence of Bacillus subtilis levansucrase gene; production of transgenic plants that express Bacillus licheniformis alpha-amylase; nucleotide sequences of tomato invertase genes; site-directed mutagenesis of barley alpha-amylase gene; maize endosperm starch branching enzyme II; WO1999/10498 the entire disclosure of which is herein incorporated by reference (improved digestibility and/or starch extraction through modification of UDP-D-xylose 4-epimerase, Fragile 1 and 2, Ref1, HCHL, C4H); U.S. Pat. No. 6,232,529 the entire disclosure of which herein incorporated by reference (method of producing high oil seed by modification of starch levels (AGP)). The fatty acid modification genes mentioned herein may also be used to affect starch content and/or composition through the interrelationship of the starch and oil pathways; altered antioxidant content or composition, such as alteration of tocopherol or tocotrienols. For example, see, U.S. Pat. No. 6,787,683, US2004/0034886, and WO2000/68393 the entire disclosures of which are herein incorporated by reference involving the manipulation of antioxidant levels and WO2003/082899 through alteration of a homogentisate geranyl geranyl transferase (hggt); altered essential seed amino acids. For example, see, U.S. Pat. No. 6,127,600 the entire disclosure of which is herein incorporated by reference (method of increasing accumulation of essential amino acids in seeds), U.S. Pat. No. 6,080,913 the entire disclosure of which is herein incorporated by reference (binary methods of increasing accumulation of essential amino acids in seeds), U.S. Pat. No. 5,990,389 the entire disclosure of which is herein incorporated by reference (high lysine), WO1999/40209 the entire disclosure of which is herein incorporated by reference (alteration of amino acid compositions in seeds), WO1999/29882 the entire disclosure of which is herein incorporated by reference (methods for altering amino acid content of proteins), U.S. Pat. No. 5,850,016 the entire disclosure of which is herein incorporated by reference (alteration of amino acid compositions in seeds), WO1998/20133 the entire disclosure of which is herein incorporated by reference (proteins with enhanced levels of essential amino acids), U.S. Pat. No. 5,885,802 the entire disclosure of which is herein incorporated by reference (high methionine), U.S. Pat. No. 5,885,801 the entire disclosure of which is herein incorporated by reference (high threonine), U.S. Pat. No. 6,664,445 the entire disclosure of which is herein incorporated by reference (plant amino acid biosynthetic enzymes), U.S. Pat. No. 6,459,019 the entire disclosure of which is herein incorporated by reference (increased lysine and threonine), U.S. Pat. No. 6,441,274 the entire disclosure of which is herein incorporated by reference (plant tryptophan synthase beta subunit), U.S. Pat. No. 6,346,403 the entire disclosure of which is herein incorporated by reference (methionine metabolic enzymes), U.S. Pat. No. 5,939,599 the entire disclosure of which is herein incorporated by reference (high sulfur), U.S. Pat. No. 5,912,414 the entire disclosure of which is herein incorporated by reference (increased methionine), WO1998/56935 the entire disclosure of which is herein incorporated by reference (plant amino acid biosynthetic enzymes), WO1998/45458 the entire disclosure of which is herein incorporated by reference (engineered seed protein having higher percentage of essential amino acids), WO1998/42831 the entire disclosure of which is herein incorporated by reference (increased lysine), U.S. Pat. No. 5,633,436 the entire disclosure of which is herein incorporated by reference (increasing sulfur amino acid content), U.S. Pat. No. 5,559,223 the entire disclosure of which is herein incorporated by reference (synthetic storage proteins with defined structure containing programmable levels of essential amino acids for improvement of the nutritional value of plants), WO1996/01905 the entire disclosure of which is herein incorporated by reference (increased threonine), WO1995/15392 the entire disclosure of which is herein incorporated by reference (increased lysine), US2003/0163838, US2003/0150014, US2004/0068767, U.S. Pat. No. 6,803,498, WO2001/79516 the entire disclosures of which are herein incorporated by reference.

Polynucleotides that control male-sterility are useful in some embodiments. There are several methods of conferring genetic male sterility available, such as multiple mutant genes at separate locations within the genome that confer male sterility, as disclosed in U.S. Pat. Nos. 4,654,465 and 4,727,219 the entire disclosures of which are herein incorporated by reference, and chromosomal translocations as described by Patterson in U.S. Pat. Nos. 3,861,709 and 3,710,511 the entire disclosures of which are herein incorporated by reference. In addition to these methods, U.S. Pat. No. 5,432,068 the entire disclosure of which is herein incorporated by reference, describe a system of nuclear male sterility which includes: identifying a gene which is critical to male fertility; silencing this native gene which is critical to male fertility; removing the native promoter from the essential male fertility gene and replacing it with an inducible promoter; inserting this genetically engineered gene back into the plant; and thus creating a plant that is male sterile because the inducible promoter is not “on” resulting in the male fertility gene not being transcribed. Fertility is restored by inducing or turning “on”, the promoter, which in turn allows the gene that confers male fertility to be transcribed; introduction of a deacetylase polynucleotide under the control of a tapetum-specific promoter and with the application of the chemical N-Ac-PPT (WO2001/29237 the entire disclosure of which is herein incorporated by reference); introduction of various stamen-specific promoters (WO1992/13956, WO1992/13957 the entire disclosures of which are herein incorporated by reference); and introduction of the barnase and the barstar polynucleotide.

For additional examples of nuclear male and female sterility systems and genes, see also, U.S. Pat. Nos. 5,859,341; 6,297,426; 5,478,369; 5,824,524; 5,850,014 and 6,265,640, all of which are hereby incorporated by reference in their entireties.

Polynucleotides that affect abiotic stress resistance including but not limited to flowering, ear and seed development, enhancement of nitrogen utilization efficiency, altered nitrogen responsiveness, drought resistance or tolerance, cold resistance or tolerance and salt resistance or tolerance and increased yield under stress are also useful in the embodiments. For example, see: WO2000/73475 the entire disclosure of which is herein incorporated by reference where water use efficiency is altered through alteration of malate; U.S. Pat. Nos. 5,892,009, 5,965,705, 5,929,305, 5,891,859, 6,417,428, 6,664,446, 6,706,866, 6,717,034, 6,801,104, WO2000/060089, WO2001/026459, WO2001/035725, WO2001/034726, WO2001/035727, WO2001/036444, WO2001/036597, WO2001/036598, WO2002/015675, WO2002/017430, WO2002/077185, WO2002/079403, WO2003/013227, WO2003/013228, WO2003/014327, WO2004/031349, WO2004/076638, WO199809521 the entire disclosures of which are herein incorporated by reference; WO199938977 the entire disclosure of which is herein incorporated by reference describing genes, including CBF genes and transcription factors effective in mitigating the negative effects of freezing, high salinity and drought on plants, as well as conferring other positive effects on plant phenotype; US2004/0148654 and WO2001/36596 the entire disclosures of which are herein incorporated by reference where abscisic acid is altered in plants resulting in improved plant phenotype such as increased yield and/or increased tolerance to abiotic stress; WO2000/006341, WO2004/090143, U.S. Pat. Nos. 7,531,723 and 6,992,237 the entire disclosures of which are herein incorporated by reference where cytokinin expression is modified resulting in plants with increased stress tolerance, such as drought tolerance, and/or increased yield. Also see, WO2002/02776, WO2003/052063, JP2002/281975, U.S. Pat. No. 6,084,153, WO2001/64898, U.S. Pat. No. 6,177,275 and U.S. Pat. No. 6,107,547 the entire disclosures of which are herein incorporated by reference (enhancement of nitrogen utilization and altered nitrogen responsiveness); for ethylene alteration, see, US2004/0128719, US2003/0166197 and WO2000/32761 the entire disclosures of which are herein incorporated by reference; for plant transcription factors or transcriptional regulators of abiotic stress, see, e.g., US2004/0098764 or US2004/0078852 the entire disclosures of which are herein incorporated by reference; polynucleotides that encode polypeptides that increase expression of vacuolar pyrophosphatase such as AVP1 (U.S. Pat. No. 8,058,515 the entire disclosure of which is herein incorporated by reference) for increased yield; nucleic acid encoding a HSFA4 or a HSFA5 (Heat Shock Factor of the class A4 or A5) polypeptides, an oligopeptide transporter protein (OPT4-like) polypeptide; a plastochron2-like (PLA2-like) polypeptide or a Wuschel related homeobox 1-like (WOX1-like) polypeptide (US2011/0283420 the entire disclosure of which is herein incorporated by reference); down regulation of polynucleotides encoding poly (ADP-ribose) polymerase (PARP) proteins to modulate programmed cell death (U.S. Pat. No. 8,058,510 the entire disclosure of which is herein incorporated by reference) for increased vigor; polynucleotide encoding DTP21 polypeptides for conferring drought resistance (US2011/0277181 the entire disclosure of which is herein incorporated by reference); nucleotide sequences encoding ACC Synthase 3 (ACS3) proteins for modulating development, modulating response to stress, and modulating stress tolerance (US2010/0287669 the entire disclosure of which is herein incorporated by reference); polynucleotides that encode proteins that confer a drought tolerance phenotype (DTP) for conferring drought resistance (WO2012/058528 the entire disclosure of which is herein incorporated by reference); tocopherol cyclase (TC) polynucleotides for conferring drought and salt tolerance (US 2012/0272352 the entire disclosure of which is herein incorporated by reference); polynucleotides encoding CAAX amino terminal family proteins for stress tolerance (U.S. Pat. No. 8,338,661 the entire disclosure of which is herein incorporated by reference); mutations in the SAL1 encoding polypeptides have increased stress tolerance, including increased drought resistant (US2010/0257633 the entire disclosure of which is herein incorporated by reference); expression of a polynucleotide encoding a polypeptide selected from the group consisting of: GRF polypeptide, RAA1-like polypeptide, SYR polypeptide, ARKL polypeptide, and YTP polypeptide increasing yield-related traits (US2011/0061133 the entire disclosure of which is herein incorporated by reference); modulating expression in a plant of a polynucleotide encoding a Class III Trehalose Phosphate Phosphatase (TPP) polypeptide for enhancing yield-related traits in plants, particularly increasing seed yield (US2010/0024067 the entire disclosure of which is herein incorporated by reference).

Other polynucleotides and transcription factors that affect plant growth and agronomic traits such as yield, flowering, plant growth and/or plant structure, can be introduced or introgressed into plants, see e.g., WO1997/49811 the entire disclosure of which is herein incorporated by reference (LHY), WO1998/56918 the entire disclosure of which is herein incorporated by reference (ESD4), WO1997/10339 and U.S. Pat. No. 6,573,430 the entire disclosures of which are herein incorporated by reference (TFL), U.S. Pat. No. 6,713,663 the entire disclosure of which is herein incorporated by reference (FT), WO1996/14414 (CON), WO1996/38560, WO2001/21822 the entire disclosures of which are herein incorporated by reference (VRN1), WO2000/44918 the entire disclosure of which is herein incorporated by reference (VRN2), WO1999/49064 the entire disclosure of which is herein incorporated by reference (GI), WO2000/46358 the entire disclosure of which is herein incorporated by reference (FR1), WO1997/29123, U.S. Pat. No. 6,794,560, U.S. Pat. No. 6,307,126 the entire disclosures of which are herein incorporated by reference (GAI), WO1999/09174 the entire disclosure of which is herein incorporated by reference (D8 and Rht) and WO2004/076638 and WO2004/031349 the entire disclosure of which is herein incorporated by reference (transcription factors).

Polynucleotides that confer increased yield are useful in the embodiments. For example, a transgenic crop plant transformed by a 1-AminoCyclopropane-1-Carboxylate Deaminase-like Polypeptide (ACCDP) coding nucleic acid, wherein expression of the nucleic acid sequence in the crop plant results in the plant's increased root growth, and/or increased yield, and/or increased tolerance to environmental stress as compared to a wild type variety of the plant (U.S. Pat. No. 8,097,769 the entire disclosure of which is herein incorporated by reference); over-expression of maize zinc finger protein gene (Zm-ZFP1) using a seed preferred promoter has been shown to enhance plant growth, increase kernel number and total kernel weight per plant (US2012/0079623 the entire disclosure of which is herein incorporated by reference); constitutive over-expression of maize lateral organ boundaries (LOB) domain protein (Zm-LOBDP1) has been shown to increase kernel number and total kernel weight per plant (US2012/0079622 the entire disclosure of which is herein incorporated by reference); enhancing yield-related traits in plants by modulating expression in a plant of a nucleic acid encoding a VIM1 (Variant in Methylation 1)-like polypeptide or a VTC2-like (GDP-L-galactose phosphorylase) polypeptide or a DUF1685 polypeptide or an ARF6-like (Auxin Responsive Factor) polypeptide (WO2012/038893 the entire disclosure of which is herein incorporated by reference); modulating expression in a plant of a nucleic acid encoding a Ste20-like polypeptide or a homologue thereof gives plants having increased yield relative to control plants (EP2431472 the entire disclosure of which is herein incorporated by reference); and polynucleotides encoding nucleoside diphosphatase kinase (NDK) polypeptides and homologs thereof for modifying the plant's root architecture (US2009/0064373 the entire disclosure of which is herein incorporated by reference).

Polynucleotides that confer plant digestibility are also useful in the embodiments. For example, altering the level of xylan present in the cell wall of a plant can be achieved by modulating expression of xylan synthase (See, e.g., U.S. Pat. No. 8,173,866 the entire disclosure of which is herein incorporated by reference).

Hordothionin protein modifications are described in U.S. Pat. Nos. 5,703,049, 5,885,801, 5,885,802, and 5,990,389, the entire disclosures of which are herein incorporated by reference. Another example is lysine and/or sulfur rich seed protein encoded by the soybean 2S albumin described in U.S. Pat. No. 5,850,016, incorporated by reference herein in its entirety and the chymotrypsin inhibitor from barley.

Insect resistance genes may encode resistance to pests that have great yield drag such as rootworm, cutworm, European corn borer, and the like. Such genes include, for example, Bacillus thuringiensis toxic protein genes (U.S. Pat. Nos. 5,366,892; 5,747,450; 5,736,514; 5,723,756; 5,593,881, the disclosure of each of which is incorporated by reference herein in its entirety). Genes encoding disease resistance traits include detoxification genes, such as against fumonosin (U.S. Pat. No. 5,792,931 the entire disclosure of which is herein incorporated by reference); avirulence (avr) and disease resistance (R) genes (Jones et al. (1994) Science 266:789; Martin et al. (1993) Science 262:1432; and Mindrinos et al. (1994) Cell 78:1089); and the like.

Herbicide resistance traits may include genes coding for resistance to herbicides that act to inhibit the action of acetolactate synthase (ALS), in particular the sulfonylurea-type herbicides (e.g., the acetolactate synthase (ALS) gene containing mutations leading to such resistance, in particular the S4 and/or Hra mutations), genes coding for resistance to herbicides that act to inhibit action of glutamine synthase, such as phosphinothricin or basta (e.g., the bar gene); glyphosate (e.g., the EPSPS gene and the gat gene; see, for example, US2004/0082770 and WO03/092360 the entire disclosures of which are herein incorporated by reference); or other such genes known in the art. The bar gene encodes resistance to the herbicide basta, the nptII gene encodes aminoglycoside 3′-phosphotransferase and provides resistance to the antibiotics kanamycin, neomycin geneticin and paromomycin, and the ALS-gene mutants encode resistance to the herbicide chlorsulfuron.

The polyribonucleotide can be, for example, a promoter hairpin, a microRNA or a non-coding RNA. A promoter hair pin can include a double-stranded ribonucleotide structure such as a stem-loop structure or an inverted-repeated sequence that may be involved in RNA interference (RNAi) or small interfering RNA (siRNA). Examples of hairpin promoters are described in, for example, in US2007/0199100, the entire disclosure of which is herein incorporated by reference.

### Methods

Methods are also provided for increasing the proportion of plants containing cells having a single copy of a polypeptide or polyribonucleotide in a population of transgenic plants. The methods include introducing the constructs described herein into a plurality of plant cells to produce a population of transgenic plants. The recombinase is expressed in the plant cells and the resulting population of transgenic plants comprises a higher number of plants containing cells having a single copy of the polypeptide or polyribonucleotide of interest compared with control plants transformed with a control vector.

The control vector is a vector that is comparable to the vectors described herein, but which lacks a component which prevents activity of the recombinase in the transformed cell. For example, the control vector may not contain the recombinase coding sequence, one or more of the recombinase target sites, or any combination thereof.

The methods result in a higher number of transformed plants containing cells having a single copy of the polypeptide or polyribonucleotide of interest. This higher number can be expressed as a single copy transformation ratio. When immature embryos are transformed, the number of single copy transformants derived from transformed immature embryos compared to the total number of immature embryos transformed is the single copy transformation ratio. The single copy transformation ratio can be similarly calculated for other tissue or cell types transformed. Examples of tissue or cell types that may be transformed include callus tissue, regenerative tissue, in vitro cultured tissue, leaf tissue, mature seed-derived tissue, embryo tissue, root tissue, anthers, microspores, germline tissues, and meristems. The single copy transformation ratio can be at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, at least about 200%, at least about 205%, at least about 210%, at least about 215%, at least about 220%, at least about 225%, at least about 230%, at least about 235%, at least about 240%, at least about 245%, at least about 250%, at least about 255%, at least about 260%, at least about 265%, at least about 270%, at least about 275%, at least about 280%, at least about 285%, at least about 290%, at least about 295%, or at least about 300% increased when using the compositions and methods disclosed herein compared with control compositions or control methods.

In certain embodiments, the methods produce a population of transgenic plants that, compared with control transgenic plants, have an increased number of plants which do not contain vector backbone downstream or upstream of the DNA construct. The frequency of plants which do not contain vector backbone in a population of transformed plants can be at least about 105%, at least about 110%, at least about 115%, at least about 120%, at least about 125%, at least about 130%, at least about 135%, at least about 140%, at least about 145%, at least about 150%, at least about 155%, at least about 160%, at least about 165%, at least about 170%, at least about 175%, at least about 180%, at least about 185%, at least about 190%, at least about 195%, at least about 200%, at least about 205%, at least about 210%, at least about 215%, at least about 220%, at least about 225%, at least about 230%, at least about 235%, at least about 240%, at least about 245%, at least about 250%, at least about 255%, at least about 260%, at least about 265%, at least about 270%, at least about 275%, at least about 280%, at least about 285%, at least about 290%, at least about 295%, or at least about 300% increased when using the compositions and methods disclosed herein compared with control compositions or control methods.

It will be apparent to those of skill in the art that variations may be applied to the compositions and methods described herein and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention.

It is to be understood that the invention is not limited in its application to the details of construction and the arrangement of components set forth in the following description. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

It also is understood that any numerical range recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this application.

All patents and patent applications mentioned in this application are incorporated by reference herein in their entireties for all purposes. In case of conflict between the present disclosure and that of a patent or publication incorporated by reference, the present disclosure controls.

The following non-limiting examples are purely illustrative.

### EXAMPLES

### Example 1: Production of Transgenic Maize Events Via Bombardment

### Immature Embryos (IEs) as a Bombardment Target

Ears of a maize (Zea mays L.) cultivar, PHR03, were surface-sterilized for 15-20 min in 20% (v/v) bleach (5.25% sodium hypochlorite) plus 1 drop of Tween™ 20 followed by 3 washes in sterile water. Immature embryos (IEs), typically 9 to 12 days after pollination, were isolated from ears and were placed scutellum-side up in an osmotic medium containing equimolar amounts of mannitol and sorbitol to give a final concentration of 0.4 M. The embryos are bombarded with gold particles coated with DNA containing bar/moPAT or another selectable marker using a PDS-1000 He biolistic device (Bio-Rad, Inc., Hercules, Calif.) at 650-1300 psi. Between 16 h and 18 h after bombardment, the bombarded embryos were placed on green tissue induction medium without osmoticum and grown at 26±2° C. under dim light (10-50 μE m⁻² s⁻¹). Following the initial 4- to 10-day culturing period, each green tissue was broken into 1 to 3 pieces depending on tissue size and transferred to green tissue induction medium supplemented with bialaphos or another selective agent. Three weeks after the first round of selection, cultures were transferred to fresh green tissue induction medium containing a selective agent at 3- to 4-week intervals. Following identification of sufficient sized green, regenerative structures, tissues were then transferred directly onto 289F maturation medium. 7-14 days of incubation on 289F regenerating shoots were transferred onto MSB rooting medium containing MS salts and vitamins, 2% sucrose, 0.25% PHYTAGEL™, 0.5 mg/L IBA and 3 mg/L bialaphos.

### Green Tissues as a Bombardment Target

Ears of PHR03 were surface-sterilized as described above. Green tissues were induced and proliferated by culturing IEs on green tissue induction medium and used for bombardment. Green tissues, approximately two- to three-months-old, were used as targets for bombardment. Tissues (4 to 6 mm) were transferred for osmotic pretreatment to green tissue induction medium containing 0.2 M mannitol and 0.2 M sorbitol. After 4 hr, tissues were bombarded as described above. Sixteen to 18 h after bombardment, the bombarded tissues were placed on green tissue induction medium without osmoticum and grown at 26±2° C. under dim light (10-50 μE m⁻² s⁻¹). Following the initial 4- to 10-day culturing period, each green tissue was broken into 1 to 3 pieces depending on tissue size and transferred to green tissue induction medium supplemented with bialaphos or another selective agent. Three weeks after the first round of selection, cultures were transferred to fresh green tissue induction medium containing a selective agent at 3- to 4-week intervals. Once transformed, transgenic green tissues are selected and cultured in a similar manner as that used for green tissue obtained by particle bombardment of immature embryos.

### Example 2: Production of Transgenic Maize Events Via Agrobacterium

### Preparation of Agrobacterium Suspension:

Agrobacterium tumefaciens harboring a binary vector containing DS-RED (RFP) reporter gene and a selectable marker (moPAT or PMI) was streaked out from a −80° frozen aliquot onto solid PHI-L medium and cultured at 28° C. in the dark for 2-3 days. PHI-L media comprised 25 ml/L stock solution A, 25 ml/L stock solution B, 450.9 ml/L stock solution C and spectinomycin added to a concentration of 50 mg/L in sterile ddH₂O (stock solution A: K₂HPO₄ 60.0 g/L, NaH₂PO₄ 20.0 g/L, adjust pH to 7.0 with KOH and autoclave; stock solution B: NH₄Cl 20.0 g/L, MgSO₄.7H₂O 6.0 g/L, KCl 3.0 g/L, CaCl₂ 0.20 g/L, FeSO₄.7H₂O 50.0 mg/L, autoclave; stock solution C: glucose 5.56 g/L, agar 16.67 g/L and autoclave). Two ways to grow Agrobacterium were used for transformation.

### A. Growing Agrobacterium on Solid Medium

A single colony or multiple colonies were picked from the master plate and streaked onto a plate containing PHI-M medium and incubated at 28° C. in the dark for 1-2 days.

Five mL Agrobacterium infection medium and 5 μL of 100 mM 3′-5′-Dimethoxy-4′-hydroxyacetophenone (acetosyringone) were added to a 14 mL Falcon tube in a hood. About 3 full loops of Agrobacterium were suspended in the tube and the tube was then vortexed to make an even suspension. One mL of the suspension was transferred to a spectrophotometer tube and the OD of the suspension was adjusted to 0.35 at 550 nm. The Agrobacterium concentration was approximately 0.5×10⁹ cfu/mL. The final Agrobacterium suspension was aliquoted into 2 mL microcentrifuge tubes, each containing 1 mL of the suspension. The suspensions were then used as soon as possible.

### B. Growing Agrobacterium on Liquid Medium

One day before infection, a 125 ml flask was set up with 30 mL of 557A with 30 μL spectinomycin (50 mg/mL) and 30 μL acetosyringone (20 mg/mL). A half loopful of Agrobacterium was suspended into the flasks and place on 200 rpm shaker at the 28° C. overnight. The Agrobacterium culture was centrifuged at 5000 rpm for 10 min. The supernatant was removed and the Agrobacterium infection medium+acetosyringone solution was added. The bacteria were resuspended by vortex and the OD of Agrobacterium suspension was adjusted to 0.35 at 550 nm.

### Maize Transformation:

Ears of a maize (Zea mays L.) cultivar, PHR03, were surface-sterilized for 15-20 min in 20% (v/v) bleach (5.25% sodium hypochlorite) plus 1 drop of Tween 20 followed by 3 washes in sterile water. Immature embryos (IEs) were isolated from ears and were placed in 2 ml of the Agrobacterium infection medium+acetosyringone solution. The optimal size of the embryos was 1.5-1.8 mm for PHR03, respectively. The solution was drawn off and 1 ml of Agrobacterium suspension was added to the embryos and the tube vortexed for 5-10 sec. The microfuge tube was allowed to stand for 5 min in the hood. The suspension of Agrobacterium and embryos were poured onto co-cultivation medium. Any embryos left in the tube were transferred to the plate using a sterile spatula. The Agrobacterium suspension was drawn off and the embryos placed axis side down on the media. The plate was sealed with PARAFILM™ tape and incubated in the dark at 21° C. for 1-3 days of co-cultivation.

Embryos were transferred to resting medium without selection. Three to 7 days later, they were transferred to green tissue induction (DBC3) medium supplemented with bialaphos or another selective agent. Three weeks after the first round of selection, cultures were transferred to fresh green tissue induction medium containing a selective agent at 3- to 4-week intervals. Once transformed, transgenic green tissues are selected and cultured in a similar manner as that used for green tissue obtained by particle bombardment of immature embryos or green tissue.

### Example 3: Vectors with Single Recombinase Site for Event Quality Improvement

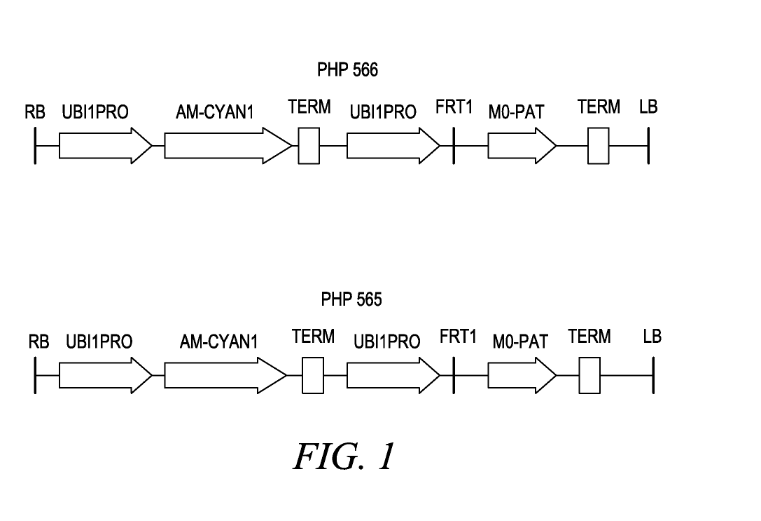

Two different JT parent binary vectors with the FLP recombinase (PHP565) and without of FLP recombinase (PHP566) was initially compared for transformation frequency and event quality using Agrobacterium strain LBA4404 in a maize cultivar, PHR03. The standard JT binary vectors without the FLP gene contains Ubi:CYAN+Ubi: FRT1-MOPAT (PHP566) while the test vector with FLP contains Ubi:FLP+Ubi: FRT1-MOPAT (PHP565) (FIG. 1); each gene has a 3′ terminator. PHP566 was designed to have the cyan fluorescent protein (CFP) to identify callus expressing CFP, and non-excision of FRT1 in plant cells. In the test vector Ubi:CFP was replaced with Ubi:FLP to demonstrate excision of the multi-copy tandem events with Ubi:FLP+Ubi: FRT1-MOPAT (FIG. 1). Transformants which are single copy events will have single copy of the T-DNA genes (FLP and MoPAT), while transformants which are multi-copy events can be identified by the presence of more than one copy of FLP, MoPAT or both genes. Table 2 shows the results of transformation frequency with PHR03. The test vector gave comparable transformation frequency as the control. The experiments were performed by two independent transformers and replicated at least twice with multiple ears.

TABLE 2
Transformation frequency and event quality frequency from
corn elite inbred line transformed with the FLP/FRT vector

| | | | TXN | Events | | MoPAT- | Quality |
|---|---|---|---|---|---|---|---|
| | #Em- | Total | % (T0 | ana- | BB− | Single | event |
| Vector | bryos | events | plant) | lyzed | % | copy | (QE) % |
| PHP566 | 953 | 341 | 36% | 247 | 78% | 47% | 41% |
| PHP565 | 929 | 286 | 31% | 194 | 85%* | 71%* | 62%* |

*Significantly different at p > 0.01

Table 2 also shows the results of the quality events. For determining the event quality, multiplex PCR assays were performed to detect the presence/absence of Agrobacterium T-DNA backbone (5 different backbone elements including VirG, VirB, Spec, LB and RB elements) while quantitative PCR was performed for determining the copy number of the MoPAT gene. Consistently, significantly higher frequency of backbone minus events (85%) was detected in the test vector PHP565; as compared to the control vector PHP566 (78%) (Table 2). This data demonstrates the ability for the FLP/FRT system to improve the frequency of backbone free events in corn. The frequency of single copy events (71%; MoPAT) was significantly higher in the events recovered from PHP565 as compared to the events recovered from control vector PHP566 (47%). Overall the quality event frequency was 1.5-fold higher in events recovered from the FLP/FRT system (Table 2).

### Example 4: Vectors with Multiple Recombinase Sites for Event Quality Improvement

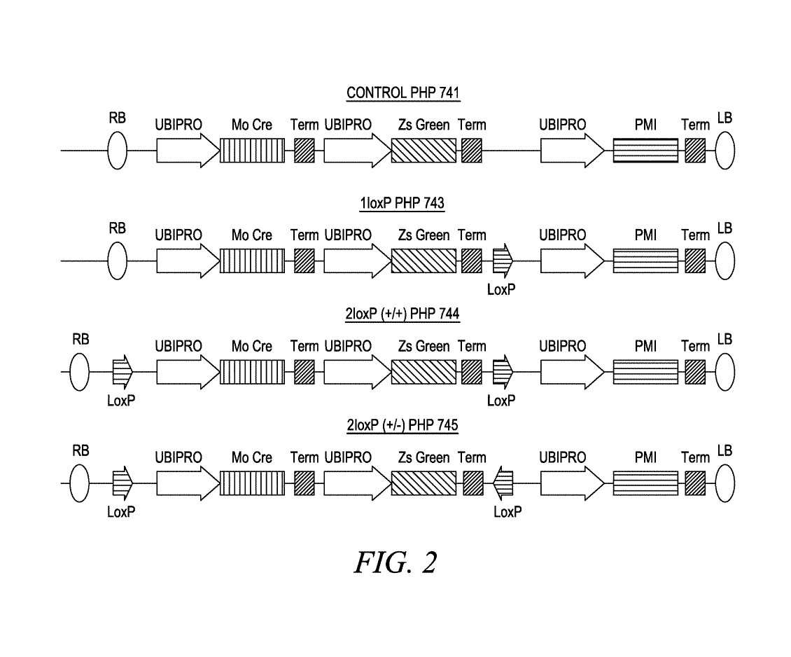

We tested an alternate recombinase system (Cre) with single and or multiple recombination recognition site (LoxP) for event quality improvement. Four different binary vectors including control (PHP741, no loxP); single loxP constructs (PHP743) and constructs with two loxP in the same orientation (PHP744) or in opposite orientations (PHP745) were constructed. All the binary vectors contained the same set of gene cassettes; Ubi:ZsGreen+Ubi: MoCre+Ubi: PMI (FIG. 2); and each gene has a 3′ terminator. The loxP site was introduced either outside the MoCre gene (PHP743; FIG. 2) or between the Ubi:ZsGreen+Ubi:MoCre expression cassette as depicted in FIG. 2 (PHP744 and PHP745). In the test vector no loxP was introduced to measure the frequency of quality events arising from standard vector, which was compared to the event quality from the loxP constructs. The quality events from PHP741 and PHP743 were identified as events which are single copy (SC) for PMI and plus events with Mo-Cre, while the quality events generated from the vectors with the multiple loxP (PHP744 and PHP445) were identified as events which are minus for MoCre and have single copy for PMI gene.

Table 3 shows the results of transformation frequency with PHR03. The test vector PHP741 with no recombination site gave very lower quality events frequency (33.4%; Single copy PMI and MoCre+), compared to the events generated from vectors transformed with a single loxP site (PHP743) which produced 58.5% quality events (Table 3). The vectors with two loxP sites, behaved quite differently when compared to each other. Both vectors PHP744 (2 loxP+/+orientation) and PHP745 (2 loxP+/−orientation) gave significantly higher quality events (72.5% and 51.4%, Table 3) as compared to the control PHP741. The vector PHP744 with two loxPs in the same orientation was found to the best vector design for improving single copy, backbone minus events. The ubiquitous expression of the Cre recombinase likely resolved tandem multi copy events either prior to integration or post integration. The experiments were performed by two independent transformers and replicated at least twice with multiple ears.

TABLE 3
The event quality frequency from PHR03
transformed with the Cre/loxP vectors.

| | Total | | | SC PMI/ | SC PMI/ | |
|---|---|---|---|---|---|---|
| Vector | Event | BB− | % | MoCre+ | MoCre− | QE % |
| PHP741 | 81 | 75 | 92.6% | 27 | | 33.3% |
| (Control) | | | | | | |
| PHP743 (1 | 106 | 98 | 92.4% | 62 | | 58.5%* |
| LoxP) | | | | | | |
| PHP744 | 138 | 134 | 97.1%* | 86.23% | 100 | 72.5%* |
| (+/+LoxP) | | | | | | |
| PHP745 | 74 | 74 | 100%* | 39.19% | 38 | 51.3%* |
| (+/−LoxP) | | | | | | |

*Significantly different at p > 0.001;
“SC” denotes Single Copy;
“BB−” denotes Backbone Minus

Table 3 also shows the results of Agro backbone minus events. The data suggested that the two loxP constructs significantly improved the backbone minus events which ranged from 97% to 100% as compared to the control. This data demonstrates the ability for the Cre/LoxP system to improve the frequency of backbone free events in corn. Based on the data from example 1 and 2, we conclude that introducing a recombinase site along with the recombinase gene cassette can significantly improve generation of backbone free events. For determining the event quality multiplex PCR assays were performed to detect the presence/absence of backbone (5 different backbone elements including Spec, LB and RB elements) while quantitative PCR was performed for determining the copy number of the PMI gene. Overall the quality event frequency was 2.0-fold or greater depending on the configuration of the loxP site in context of the Cre recombinase cassette in the vector (Table 3, FIG. 2).

### Example 5. Vectors with Overdrive and Overdrive Plus Multiple Recombinase Sites for Event Quality Improvement

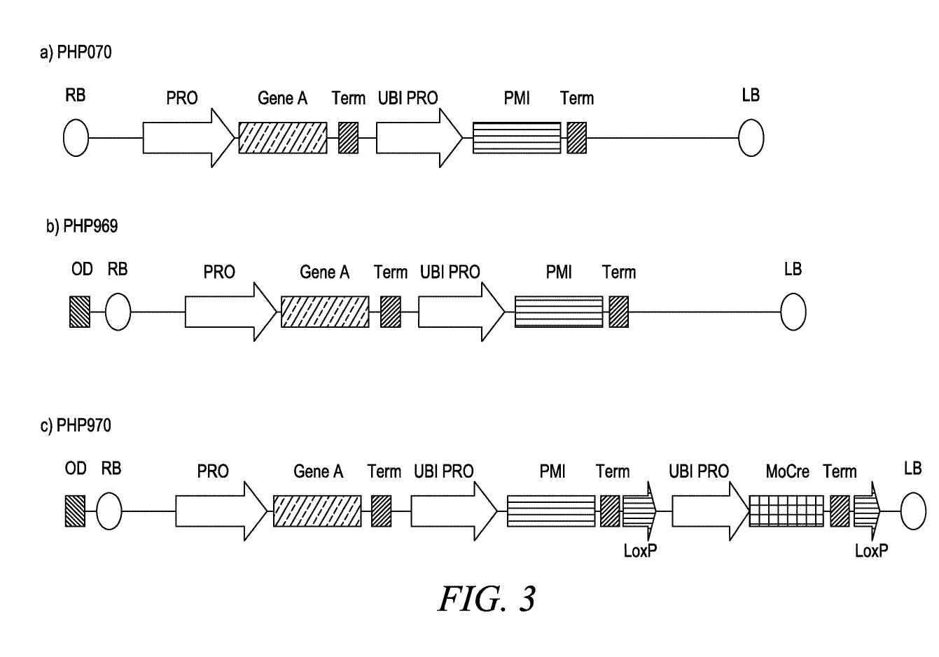

Vectors were designed to test the effects of the Overdrive sequence (OD, a cis acting element; Peralta et. al. 1986) and OD plus a recombinase system (Cre) with multiple recognition sites (LoxP) for event quality improvement. Three different binary vectors; control (PHP070, no MoCre and loxP); OD (PHP969; no MoCre and loxP) and OD+Cre (PHP970; with MoCre and loxP sites in direct orientation) were constructed (FIG. 3). All the binary vectors contained a stack of trait genes and Ubi: PMI: PINII as the selectable marker. The quality events from PHP070 and PHP969 were identified as events which are single copy (SC) for all the trait genes, and PMI without backbone vector insertion. We identified the quality events for the MoCre excision vector as events which were minus for MoCre with single copy of all trait genes, PMI gene and free of backbone insertion.

TABLE 4
The event quality frequency from PHR03 transformed
with the Cre/loxP vectors with and without OD.

| | | | Single | | | |
|---|---|---|---|---|---|---|
| | | | copy and | | | |
| | Single | | backbone | | Multi- | |
| | Copy | Single | free | Quality | copy | Multi- |
| Vector | events | Copy % | events | event | events | copy |
| 070 | 150 | 44.2% | 140 | 41.3% | 131 | 36.1% |
| (Control) | | | | | | |
| 969 (OD) | 143 | 53.4%* | 133 | 49.6% | 76 | 25.7% |
| 970 | 133 | 65.2%* | 123 | 60.3% | 35 | 15.2% |
| (OD + | | | | | | |
| MoCRE + | | | | | | |
| LoxP) | | | | | | |

*Significantly different at p > 0.005

For determining the event quality, multiplex PCR assays were performed to detect the presence/absence of backbone (5 different backbone elements including Spec, LB and RB elements) while quantitative PCR was performed for determining the copy number of the trait gene stack and PMI gene. Table 4 shows the results of quality event frequency with OD and OD+Cre vectors. The data showed that the construct with OD significantly reduced the frequency of multiple copy events (1.4×), improving the overall quality event frequency by 1.2-fold. Suggesting the Overdrive element reduces production of multi-copy events in corn, enriching the recovery of single copy events. Similarly with the OD+MoCre construct, we observed a significant reduction in the multi-copy events (2.4×). The data also demonstrates the use of OD and MoCre could significantly improve recovery of single copy events by 1.5× compared to the controls. This data further illustrates the ability for the Cre/LoxP system to improve the frequency of backbone free, single copy events in corn as mentioned in the earlier example (Example 4). Based on the data from example 3, 4 and 5, we conclude that introducing a recombinase site along with the recombinase gene cassette can significantly improve generation of quality events in plants.

### Example 6. Natural Desiccation and Gene Excision in Transgenic Mature Maize and Improvement of Quality Event Ratio in T1 Progeny Plants

### Maize Transformation:

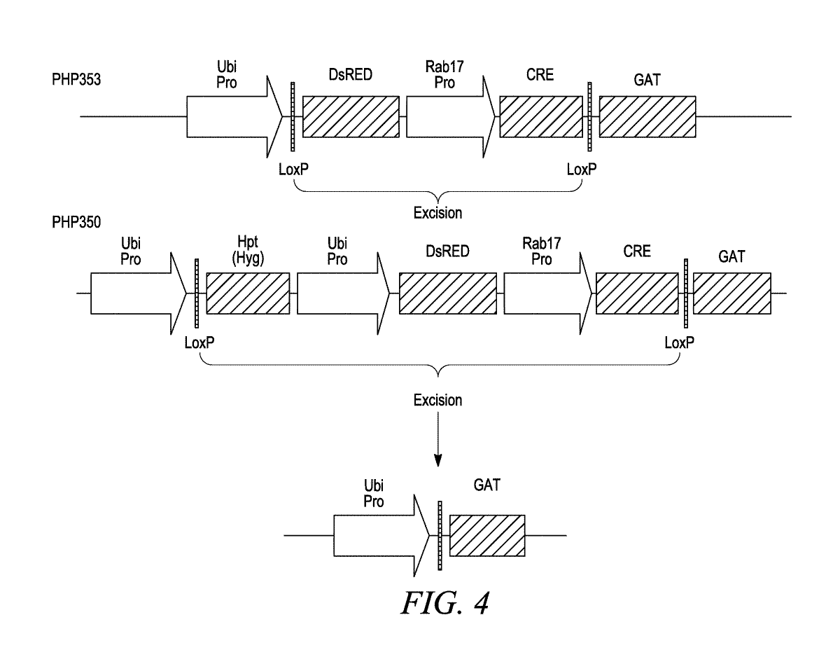

A maize elite inbred, PHR03, was transformed with AGL1/PHP353 as described in FIG. 4. Immature embryos from maize inbred PHR03 were harvested 9-13 days post-pollination with embryo sizes ranging from 1.3-2.2 mm length and were co-cultivated with AGL1/PHP353 (an excision vector) on PHI-T medium for 3 days in dark conditions. These embryos were then transferred to DBC3 medium containing 100 mg/L cefotaxime in dim light conditions. After 2-3 weeks RFP-expressing sectors were picked up and proliferated on the same medium. When the tissue amount of each transgenic event was sufficient, tissues were moved to PHI-RF maturation medium. Regenerating shoots were transferred to MSB medium in PHYTATRAYs™ containing 100 mg/L cefotaxime for rooting. Plants with good roots were transferred to soil for further growth, glyphosate spray test and molecular assay.

### Glyphosate Resistance Confirmation:

To confirm that the natural desiccation process that occurs during seed maturation would in fact allow for the excision of DsRed and resistance to glyphosate, seeds collected from T₀ plants crossed with wild-type PHR03 pollen were germinated in soil. By planting seeds straight to soil without any treatments, excision would be a result of natural processes.

Eight random events were chosen to be tested by this method. About twenty mature T₁ seeds each from the following 8 events, PHP353 T0 event #s 4, 5, 6, 7, 10, 11, 13 and 14 were placed in small pots with METRO MIX™ soil (Sun Gro Horticulture, McFarland, Calif.) with fertilizer and placed in the greenhouse. After plants had germinated and grown to about 12-18 cm (10-12 days after planting), the plants were then sprayed with glyphosate+surfactant at 1× or 2× concentration; 1× is equivalent to what is used in the field. Before spraying, all pots were evenly spaced and positioned to ensure that they would receive an even distribution of glyphosate. The distance between the sprayer nozzle and the apical meristem of the plants was approximately 18 inches. Within 8-10 days, it was visibly evident which plants were not affected by the herbicide and which plants had been severely damaged.

The results of the spray test are presented in Table 5. From visible spray test results, all wild-type PHR03 plants had been severely damaged, as predicted. It was also clear that some plants from event #s 6, 7, 10, 13 and 14 had small damage or no signs of damage and continued to grow at a normal rate having not lost any leaf tissue (Table 5). However, all plants from event #s 4, 5 and 11 showed damage equivalent to that of the wild-type PHR03 plants, which was not expected.

### Improvement of Quality Event Ratio in T1 Progeny Plants by Gene Excision:

The surviving individual T1 plants after glyphosate spray were analyzed for copy number by qPCR. As shown in Table 5, we could further improve quality event (single copy event without the Agrobacterium backbone sequence) ratio through gene excision in T1 progeny plants. One event, event #10 was already pre-excised in T0 plants and only GAT was present possibly due to too strong expression CRE by the maize Rab17 promoter or facing desiccated conditions during plant culturing/growth. Thirty-three % (⅓) of T0 discard (Agro backbone-positive or multi-copy) events generated quality plants in T1 progeny plants germinated from mature seed

TABLE 5
Glyphosate Spray Test on Plants Germinated from T1
Mature Maize Seed and Copy Number Assay Results

| | | | | Glyphosate | |
|---|---|---|---|---|---|
| Event | T-DNA | DS-RED | GAT | Resistance | Quality |
| # | Backbone | qPCR | qPCR | in T1 | Events T1 |
| 4 | POSITIVE | 2 | 2 | Susceptible | |
| 5 | NEGATIVE | 1 | 1 | Susceptible | |
| 6 | NEGATIVE | 1 | 2 | Resistant | Quality |
| 7 | NEGATIVE | 2 | 3 | Resistant | Multi copy |
| 10 | NEGATIVE | NULL | 1 | Resistant | Quality |
| 11 | NEGATIVE | 2 | 3 | Susceptible | |
| 13 | POSITIVE | 2 | 3 | Resistant | n.d. |
| 14 | POSITIVE | 4 | 4 | Resistant | Multi copy |
| PHR03 | NEGATIVE | NULL | NULL | Susceptible | |
| Control | | | | | |

### Example 7: Agrobacterium-Mediated Transformation of Wheat Using Immature Embryos (IEs)

### Preparation of Agrobacterium Suspension:

Agrobacterium tumefaciens harboring vector of interest was streaked from a −80° frozen aliquot onto solid LB medium containing selection (kanamycin or spectinomycin). The Agrobacterium was cultured on the LB plate at 21° C. in the dark for 2-3 days. A single colony was selected from the master plate and was streaked onto an 810D medium plate containing selection and it was incubated at 28° C. in the dark overnight. A sterile spatula was used to collect Agrobacterium cells from the solid medium and cells were suspended in ˜5 mL wheat infection medium (WI4) with 400 μM acetosyringone (As) (Table 6). The OD of the suspension was adjusted to 0.1 at 600 nm using the same medium.

### Wheat Immature Embryo Transformation:

Material Preparation, Sterilization and Sand Treatment

4-5 spikes were collected containing immature seeds with 1.5-2.5 mm embryos. Immature seeds/wheat grains were then isolated from the spike by pulling downwards on the awn and removing both sets of bracts (the lemma and palea). Wheat grains were surface-sterilized for 15 min in 20% (v/v) bleach (5.25% sodium hypochlorite) plus 1 drop of Tween 20, then were washed in sterile water 2-3 times. Immature embryos (IEs) were isolated from the wheat grains and were placed in 1.5 ml of the WI4 medium in 2 mL microcentrifuge tubes. For sand treatments, IEs were isolated and placed in 1 mL of WI4 medium with 0.25 mL of autoclaved sand. The 2 mL microcentrifuge tubes containing the IEs were centrifuged at 6 k for 30 seconds, vortexed at 4.5, 5 or 6 for 10 seconds, and then centrifuged at 6 k for 30 seconds. Embryos stood in tubes for 20 minutes.

Embryo Treatments with Sand and Infection

WI4 medium was drawn off, and 1.0 ml of Agrobacterium suspension was added to the 2 mL microcentrifuge tubes containing the IEs. Embryos were left in tubes for 20 minutes. The suspension of Agrobacterium and IEs was poured onto wheat co-cultivation medium, WC21 (Table 7). Any embryos left in the tube were transferred to the plate using a sterile spatula. The IEs were placed embryo axis side down on the media and it was ensured that the embryos were immersed in the solution. The plate was sealed with PARAFILM™ tape and incubated in the dark at 25° C. for 3 days of co-cultivation.

### Media Scheme and Selection

After 3 days of co-cultivation IEs were transferred embryo axis side down to DBC4 green tissue (GT) induction medium containing 100 mg/L cefotaxime (PhytoTechnology Lab., Shawnee Mission, Kans.) (Table 8). All embryos were then incubated at 26-28° C. in dim light for two weeks, then were transferred to DBC6 tissue (GT) induction medium containing 100 mg/L cefotaxime for another two weeks (Table 9). Regenerable sectors appear 3-4 weeks after transformation and will be ready for regeneration after being isolated. Regenerable sectors were cut from the non-transformed tissues and placed on regeneration media MSA with 100 mg/L cefotaxime (Table 10). Sectors on MSA medium should be placed in bright light for 1.5-2 weeks or until roots and elongated shoots have formed. After sectors have developed into small plantlets they were transferred to PHYTATRAYs™ until plantlets are ready to be transferred to soil. During each transfer plantlets were checked for marker gene expression and any non-expressing or chimeric tissues were removed.

TABLE 6
Liquid Wheat Infection (WI4) Medium

| DI water | 1000 | mL |
|---|---|---|
| MS salt + Vitamins | 4.43 | g |
| Maltose | 30 | g |
| Glucose | 10 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| Adjust PH to 5.8 with KOH | | |
| Post sterilization | | |
| Acetosyringone (1M) | 400 | μl |

TABLE 7
Wheat Co-cultivation (WC21) Medium

| DI water | 1000 | mL |
|---|---|---|
| MS salt + Vitamins | 4.43 | g |
| Maltose | 30 | g |
| MES | 1.95 | g |
| 2,4-D (0.5 mg/L) | 1 | ml |
| Picloram (10 mg/ml) | 200 | μl |
| BAP (1 mg/L) | 0.5 | ml |
| 50X CuSO4 (0.1M) | 49 | μl |
| Adjust PH to 5.8 with KOH | | |
| Add 3.5 g/L of Phytagel | | |
| Post sterilization | | |
| Acetosyringone (1M) | 400 | μl |

TABLE 8
DBC4 Medium

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 2 | mL |
| BAP | 1 | mL |
| Adjust PH to 5.8 with KOH | | |
| Add 3.5 g/L of Phytagel | | |
| Post sterilization | | |
| Cefotaxime (100 mg/ml) | 1 | ml |

TABLE 9
DBC6 Medium

| dd H20 | 1000 | mL |
|---|---|---|
| MS salt | 4.3 | g |
| Maltose | 30 | g |
| Myo-inositol | 0.25 | g |
| N-Z-Amine-A | 1 | g |
| Proline | 0.69 | g |
| Thiamine-HCl (0.1 mg/mL) | 10 | mL |
| 50X CuSO4 (0.1M) | 49 | μL |
| 2,4-D (0.5 mg/mL) | 1 | mL |
| BAP | 2 | mL |
| Adjust PH to 5.8 with KOH | | |
| Add 3.5 g/L of Phytagel | | |
| Post sterilization | | |
| Cefotaxime (100 mg/ml) | 1 | ml |

TABLE 10
Regeneration MSA Medium

| dd H20 | 1000 | mL |
|---|---|---|
| MS salt + Vitamins(M519) | 4.43 | g |
| Sucrose | 20 | g |
| Myo- Inositol | 1 | g |
| Adjust PH to 5.8 with KOH | | |
| Add 3.5 g/L of Phytagel | | |
| Post sterilization | | |
| Cefotaxime (100 mg/ml) | 1 | ml |

### Example 8: Gene Excision Induction and Plant Regeneration from Desiccated T₁ Immature Wheat Embryos

### Wheat Transformation and Immature Embryos Isolation:

Excision vectors, AGL1/PHP350 and AGL1/PHP353, were used for wheat (cv. Fielder) transformation. Wheat transformation was performed and T₁ immature embryos (IEs) with 2.0-3.0 mm from transgenic plants were isolated as described in Example #1.

### Desiccation, Selection and Regeneration:

Sterilized IEs were placed scutellum side down on sterile fiber glass filter paper in a Petri dish. 300 μL of DBC6 liquid medium was added to the filter paper to prevent over drying. Plates were wrapped with PARAFILM™ and checked for expression of DsRed from PHP350 and PHP353 before desiccation in order to compare expression after desiccation. Plates were moved into a sterile laminar hood unwrapped and stood for 2-4 days until the embryos appeared darker and shrunken, and were desiccated. Embryos were then placed scutellum side down on to DBC6 GT induction medium or MSA regeneration medium containing 100 mg/L cefotaxime and with 30 or 50 μM glyphosate for selection. Five to 10 days later DsRed expression was checked in the emerging shoots. Embryos that had been properly desiccated had very weak or no DsRed expression as the gene was excised via the LoxP sites. Both transgenic and nontransgenic embryos without desiccation treatment germinated well on glyphosate-free medium while both of them had completely inhibited germination on 30 μM glyphosate. Embryos that successfully underwent gene excision by desiccation had glyphosate resistance and regenerated on medium containing 30 to 50 μM glyphosate.

Healthy plantlets were transferred to MSA medium in PHYTATRAYs™ containing 100 mg/L cefotaxime and 30 or 50 μM glyphosate for further selection and growth.

### Example 9: Natural Desiccation and Gene Excision in Transgenic Mature Wheat and Improvement of Quality Event Ratio in T1 Progeny Plants

An alternative way to conduct desiccation treatment on wheat transformed with excision vectors, AGL1/PHP350 and AGL1/PHP353, was by natural desiccation in mature T₁ seed.

### PHP350 and PHP353 Transgenic Mature Seed Excision:

### Mature Seed Sterilization

T1 mature seed transformed with AGL1/PHP350 and/or AGL1/PHP353 were placed in a 100×15 mm petri dish and laid in a single layer (maximum approximately 115 seeds/plate). Two to four plates were placed in a bell jar desiccator within a fume hood. To ensure that all surfaces of the plates were exposed, seeds were positioned in a single layer manner that would also accommodate a 250 mL beaker. The 250 mL beaker was filled with 100 mL of bleach, then 3.5 mL of 12 N HCl was added drop wise along the side of the beaker. The desiccator jar was closed immediately and left to stand overnight (max 16 hours). After overnight exposure to chlorine gas, petri dishes were closed and moved to laminar flow hood. In the laminar flow hood the plates were opened, allowing them to air out for approximately 30 minutes to remove excess chlorine gas.

### Selection/Regeneration

Sterilized seeds were then transferred, embryo side up, to DBC6 or MSA medium containing 100 mg/L cefotaxime with 30 or 50 μM glyphosate for selection. After 5-10 days DsRed expression was checked in the emerging shoots; seeds that had been excised no longer had DsRed expression as the gene was cleaved via the Lox P sites. Those seeds that were successfully excised of DsRed had glyphosate resistance and regenerate on medium containing glyphosate. Once seeds had healthy shoot and root formation, the plantlets were moved to MSA medium containing 100 mg/L cefotaxime in PHYTATRAYs™ with 30 or 50 μM glyphosate for selection.

### Glyphosate Resistance Confirmation

To confirm that the natural desiccation process of seed maturation would in fact allow for the excision of DsRed and resistance to glyphosate, seeds collected from T0 plants were germinated in soil. By planting seeds straight to soil without any treatments the method of excision would truly be natural.

Twenty-one random events were chosen to be tested by this method. About 20 seeds from each event transformed with PHP350 or PHP353 were placed in small pots with metro mix soil with fertilizer and placed in a growth chamber. After plants had germinated and grown to about 19-24 cm they were moved to 1 gallon pots and allowed to acclimate for 3-4 days and then moved to the greenhouse. Before the glyphosate spray, leaf punch samples were harvested for DNA extraction.

All pots, including wild-type Fielder plants, were then sprayed with TOUCHDOWN™ glyphosate+surfactant at 2× or 4× concentrations which is equivalent to what is used in the field. Before spraying, all pots were evenly spaced and positioned to ensure that they would receive an even distribution of glyphosate. The distance between the sprayer nozzle and the apical meristem of the plants is approximately 18 inches. Within 10 days it was visibly evident which plants were not affected by the herbicide and which plants had been severely damaged.

From visible spray test results, all wild-type Fielder (WT) plants had been severely damaged, as predicted (Table 11). It was also clear that 81% (17/21) of 21 events tested were glyphosate-resistant in their T1 plants; 10 PHP350 (#s 3, 7-9, 12, 14-18) and 7 PHP353 events (#s 1, 3, 5-9) had no signs of damage and continued to grow at a normal rate having not lost any leaf tissue (Table 11). However, PHP350 event #5 and PHP353 events #2 and 4 showed damaged equivalent to that of the wild-type Fielder plants which was not expected until qPCR results from the T0 plants were examined. PHP350 event #1 also had very weak resistance and could not survive.

TABLE 11
Glyphosate Spray Test on Plants Geminated from
T1 Mature Seed and Copy Number Assay Results.

| | | | | | | # T1 Quality Plants/# |
|---|---|---|---|---|---|---|
| | | | | | | T1 Plants Tested for |
| | | T-DNA | DS-RED | GAT | Glyphosate | qPCR after |
| PHP | Event # | Backbone | qPCR | qPCR | Resistance | Glyphosate Spray |
| 350 | 2 | NEGATIVE | 1 | 1 | − | — |
| 350 | 3 | POSITIVE | 2 | 2 | + | 3/9 |
| 350 | 5 | NEGATIVE | 2 | NULL | − | — |
| 350 | 7 | NEGATIVE | 4 | 2 | + | 0/8 |
| 350 | 8 | NEGATIVE | 2 | 1 | + | 0/7 |
| 350 | 9 | NEGATIVE | 4 | 3 | + | 0/6 |
| 350 | 12 | NEGATIVE | 1 | 1 | + | 7/15 |
| 350 | 14 | NEGATIVE | 4 | 1 | + | 0/0 |
| 350 | 15 | NEGATIVE | 1 | 1 | + | 2/9 |
| 350 | 16 | NEGATIVE | 1 | 1 | + | 1/10 |
| 350 | 17 | POSITIVE | >4COPY | 4 | + | 0/0 |
| 530 | 18 | NEGATIVE | 3 | 1 | + | 0/0 |
| | 12 events | | | | | |
| 353 | 1 | NEGATIVE | 3 | 4 (3) | + | 0/5 |
| 353 | 2 | NEGATIVE | 1 | NULL | − | — |
| 353 | 3 | NEGATIVE | 3 | 2 | + | 1/8 |
| 353 | 4 | NEGATIVE | 1 | NULL | − | — |
| 353 | 5 | NEGATIVE | 2 | 2 | + | 2/10 |
| 353 | 6 | NEGATIVE | 2 | 2 | + | 7/7 |
| 353 | 7 | NEGATIVE | 2 | 2 | + | 0/7 |
| 353 | 8 | NEGATIVE | 4 | 4 | + | 3/6 |
| 353 | 9 | NEGATIVE | 1 | 2 | + | 3/12 |
| | 9 events | | | | | |
| WT | Control | NEGATIVE | NULL | NULL | − | |
| Fielder | | | | | | |

The qPCR results indicated that all glyphosate-resistant events and PHP350 event #1 had both DsRed and GAT genes in T0 plants; conversely PHP350 event #5 and PHP353 events #2 and 4 did not have the GAT gene (Table 11). Because the T0 plant from these events did not have the GAT gene, the T1 seeds also did not have the GAT gene. Therefore even with the excision of DsRed at the LoxP sites there could not be glyphosate resistance without the presence of the GAT gene.

Improvement of Quality Event Ratio in T1 Progeny Plants by Gene Excision: The surviving individual T1 plants after glyphosate spray were analyzed for copy number by qPCR. As shown in Table 7, we could further improve quality event (single copy event without the Agrobacterium backbone sequence) ratio through gene excision in T1 progeny plants. 55% (6/11) of T0 “discard” (Agro backbone-positive or multi-copy) events generated quality plants in T1 progeny plants germinated from mature seed. The interpretation for this is as follows: multiple copies with tandem repeats or reverse orientation (intact or truncated) will be excised by CRE (or other recombinase) gene expression; multiple copy genes will be eliminated if the Lox sites (or other recombination sites) is in the right orientation for functionality regardless of the # of Lox-Lox sites in multiple copy events. However, when transgenes are integrated at 2 or more different chromosomes, T0 plants will still show multiple copies even after gene excision. For example, the transgenic events with a single copy on chromosome 1 and another single copy (or multiple copies)+1−Agrobacterium backbone on chromosome 2 before or even after gene excision will be considered as multiple copy events+/−Agrobacterium backbone (not quality events) (Table 12). However, we could obtain quality T1 progeny plants with a single copy minus Agrobacterium backbone from this event by segregation. Detailed copy number assay results from 7 T1 discard events are shown in Table 13.

TABLE 12
Copy Number Assay Results from a Gene Excision Event.

| | | | | | | Glyph- |
|---|---|---|---|---|---|---|
| | | | | | | osate |
| Genera- | | | T-DNA | DS-RED | GAT | Toler- |
| tion | PHP | Event # | Backbone | qPCR | qPCR | ance |
| T0 parent | 350 | 3 | POSITIVE | 2 | 2 | + |
| T1 | 350 | 3-1 | NEGATIVE | NULL | 1 | + |
| T1 | 350 | 3-2 | NEGATIVE | NULL | 1 | + |
| T1 | 350 | 3-3 | NEGATIVE | NULL | 1 | + |
| T1 | 350 | 3-4 | POSITIVE | NULL | 4 | + |
| T1 | 350 | 3-5 | POSITIVE | NULL | 2 | + |
| T1 | 350 | 3-6 | POSITIVE | NULL | 1 | + |
| T1 | 350 | 3-7 | POSITIVE | NULL | 1 | + |
| T1 | 350 | 3-8 | POSITIVE | NULL | 4 | + |
| T1 | 350 | 3-9 | POSITIVE | NULL | 4 | + |

TABLE 13
T1 qPCR DATA from Wheat PHP350 and PHP353 Gene Excision

| | | | T-DNA | DS-RED | GAT | # of Transgene |
|---|---|---|---|---|---|---|
| Generation | PHP | Event # | Backbone | qPCR | qPCR | Integration Loci |
| T0 parent | 350 | 3 | POSITIVE | 2 | 2 | 2 |
| T1 | 350 | 3-1 | NEGATIVE | NULL | 1 | |
| T1 | 350 | 3-2 | NEGATIVE | NULL | 1 | |
| T1 | 350 | 3-3 | NEGATIVE | NULL | 1 | |
| T1 | 350 | 3-4 | POSITIVE | NULL | 4 | |
| T1 | 350 | 3-5 | POSITIVE | NULL | 2 | |
| T1 | 350 | 3-6 | POSITIVE | NULL | 1 | |
| T1 | 350 | 3-7 | POSITIVE | NULL | 1 | |
| T1 | 350 | 3-8 | POSITIVE | NULL | 4 | |
| T1 | 350 | 3-9 | POSITIVE | NULL | 4 | |
| T0 parent | 350 | 7 | NEGATIVE | 4 | 2 | 2 |
| T1 | 350 | 7-1 | NEGATIVE | NULL | 4 | |
| T1 | 350 | 7-2 | NEGATIVE | NULL | 4 | |
| T1 | 350 | 7-3 | NEGATIVE | 2 | 4 | |
| T1 | 350 | 7-4 | NEGATIVE | 4 | 4 | |
| T1 | 350 | 7-5 | NEGATIVE | 1 | 4 | |
| T1 | 350 | 7-6 | NEGATIVE | 2 | 4 | |
| T1 | 350 | 7-7 | NEGATIVE | 4 | 4 | |
| T1 | 350 | 7-8 | NEGATIVE | NULL | >4COPY | |
| T0 parent | 353 | 1 | NEGATIVE | 3 | 4 | 2 |
| T1 | 353 | 1-1 | NEGATIVE | 4 | 4 | |
| T1 | 353 | 1-2 | NEGATIVE | 1 | 4 | |
| T1 | 353 | 1-3 | NEGATIVE | 1 | 4 | |
| T1 | 353 | 1-4 | NEGATIVE | 4 | 4 | |
| T1 | 353 | 1-5 | NEGATIVE | 4 | 4 | |
| T0 Parent | 353 | 5 | NEGATIVE | 2 | 2 | 1 |
| T1 | 353 | 5-1 | NEGATIVE | NULL | 2 | |
| T1 | 353 | 5-2 | NEGATIVE | NULL | 1 | |
| T1 | 353 | 5-3 | NEGATIVE | 2 | 2 | |
| T1 | 353 | 5-4 | NEGATIVE | NULL | 2 | |
| T1 | 353 | 5-5 | NEGATIVE | NULL | 3 | |
| T1 | 353 | 5-6 | NEGATIVE | 1 | 2 | |
| T1 | 353 | 5-7 | NEGATIVE | NULL | 2 | |
| T1 | 353 | 5-8 | NEGATIVE | NULL | 2 | |
| T1 | 353 | 5-9 | NEGATIVE | NULL | 1 | |
| T1 | 353 | 5-10 | NEGATIVE | NULL | 2 | |
| T0 Parent | 353 | 6 | NEGATIVE | 2 | 2 | 1 |
| T1 | 353 | 6-1 | NEGATIVE | NULL | 1 | |
| T1 | 353 | 6-2 | NEGATIVE | NULL | 1 | |
| T1 | 353 | 6-3 | NEGATIVE | NULL | 1 | |
| T1 | 353 | 6-4 | NEGATIVE | NULL | 1 | |
| T1 | 353 | 6-5 | NEGATIVE | NULL | 1 | |
| T1 | 353 | 6-6 | NEGATIVE | NULL | 1 | |
| T1 | 353 | 6-7 | NEGATIVE | NULL | 1 | |
| T0 Parent | 353 | 7 | NEGATIVE | 2 | 2 | 2 |
| T1 | 353 | 7-1 | NEGATIVE | NULL | 4 | |
| T1 | 353 | 7-2 | NEGATIVE | 2 | 3 | |
| T1 | 353 | 7-3 | NEGATIVE | NULL | 3 | |
| T1 | 353 | 7-4 | NEGATIVE | NULL | 2 | |
| T1 | 353 | 7-5 | NEGATIVE | 1 | 2 | |
| T1 | 353 | 7-6 | NEGATIVE | NULL | 2 | |
| T1 | 353 | 7-7 | NEGATIVE | NULL | 4 | |
| T0 Parent | 353 | 8 | NEGATIVE | 4 | 4 | 1 |
| T1 | 353 | 8-1 | NEGATIVE | NULL | 1 | |
| T1 | 353 | 8-2 | NEGATIVE | NULL | 3 | |
| T1 | 353 | 8-3 | NEGATIVE | NULL | 2 | |
| T1 | 353 | 8-4 | NEGATIVE | NULL | 3 | |
| T1 | 353 | 8-5 | NEGATIVE | NULL | 1 | |
| T1 | 353 | 8(1)6 | NEGATIVE | NULL | 1 | |

The quality events obtained from T1 plants by gene excision was stable in transgene inheritance in their T2 generations (Table 14). 100% (7/7) of T1 quality events generated quality plants in T2 plants.

TABLE 14
Stable Transgene Inheritance of Quality Events in T₂ Plants by Gene Excision

| | | | | | Glyphosate | # T2 Single- |
|---|---|---|---|---|---|---|
| | | T-DNA | T₁ DS-RED | GAT | Resistance | Copy Quality |
| PHP # | Event # | Backbone | qPCR | qPCR | in T2 Plants | Event Plants |
| 350 | 3.3 | NEGATIVE | NULL | 1 | + | 4 out of 6 |
| 350 | 3.4 | NEGATIVE | NULL | 1 | + | 8 out of 16 |
| 350 | 12.1 | NEGATIVE | NULL | 1 | + | 1 out of 3 |
| 350 | 15.1 | NEGATIVE | NULL | 1 | + | 4 out of 8 |
| 353 | 5.1 | NEGATIVE | NULL | 1 | + | 2 out of 5 |
| 353 | 6.1 | NEGATIVE | NULL | 1 | + | 6 out of 8 |
| 353 | 8.1 | NEGATIVE | NULL | 1 | + | 1 out of 3 |

### Example 10: Soybean Transformation and Excision

We tested two promoters, constitutive (Soybean EF1A, PHP68841) or inducible (Arabidopsis Heat Shock 18.1, PHP68842) promoters to control the expression of the CRE recombinase. Identical LOXP1 recombination sites surrounding the recombinase, isopentenyl transferase (ipt), and red fluorescent protein (RFP) genes allowed excision of these intervening sequences by the CRE recombinase. The construct also contained a green fluorescent protein (ZsGreen, ZSG; Clontech, Mountain View, Calif., USA) gene that, prior to excision, lacks a promoter. Excision brings the ZsGreen coding region under control of the soybean ubiquitin promoter. Also tested was a construct without the recombinase, but having BAR and ZsYellow expression cassettes (PHP54628).

The ipt gene is from Agrobacterium and codes for an enzyme that represents a rate limiting step for cytokinin biosynthesis. Overexpression of ipt in plants is known to lead to overproduction of cytokinins and has been shown to stimulate shoot production in tissue culture (Zuo et al. (2002) Curr Opin Biotechnol 13:173-180; Ebinuma & Komamine (2001) In Vitro Cell Dev Biol Plant 37:103-113). Lack of excision of the ipt gene will lead to events that overproduce cytokinin, and therefore will remain as multiple shoot structures that cannot be regenerated. The constructs tested are as follows:

### PHP68842: LB-GmUBQ-LoxP-RFP-HSP::CRE-UBIQ10::IPT-loxP-ZsGREEN-35S::BAR-RB

### PHP68841: LB-GmUBQ-LoxP-RFP-EF1A::CRE-UBIQ10::IPT-loxP-ZsGREEN-35S::BAR-RB

### PHP54628: RB-GmSAMS::CaMV35S::BAR::Nos-GmUBQ::ZsYellow::Nos-LB

The constructs described above were introduced into soybean by infecting the cotyledonary node region of mature, hydrated seed with Agrobacterium, essentially as described in U.S. Pat. No. 7,473,822B1 (herein incorporated by reference), and Paz et al. (2006) Plant Cell Rep 25:206-213.

In studies using the heat shock promoter, following introduction of the heat shock construct, the tissue was subjected to a heat treatment at different times during the process. Petri plates containing about 6 explants each were placed in an incubator set at 42° C. for one hour. This treatment was repeated on three consecutive days and was done after the recovery, shoot initiation and shoot elongation phases of the transformation process. Transgenic plants were recovered using selection for expression of the BAR gene that confers resistance to the herbicide, Bialaphos. Transgene copy number was determined by qPCR using probes for BAR coding region, UBQ3 TERM of the RFP gene, UBQ10 promoter for IPT, and the ZsGREEN coding region. Therefore, CRE-mediated excision could be assessed and the copy number of the remaining transgenes could be determined. These results are summarized in Table 15.

TABLE 15

| Promoter | | #Explants | | Txn | Single | Single |
|---|---|---|---|---|---|---|
| (Cre) | Treatment | infected | BAR+ | Rate | Copy* | Copy |
| PHP68842 | Early HS | 276 | 23 | 8% | 16 | 70% |
| | Mid HS | 276 | 32 | 12% | 13 | 41% |
| | Late HS | 276 | 24 | 9% | 7 | 29% |
| | none | 276 | 7 | 3% | 3 | 43% |
| PHP68841 | none | 120 | 11 | 9% | 6 | 55% |
| PHP54628 | none | 84 | 3 | 4% | 1 | 33% |

*For EF1A and Heat Shock vectors, all single copy events displayed excision

The results indicate that events transformed with the heat shock excision vector generally had a high proportion of single copy events. When the heat shock treatment was applied early in the transformation process, 70% of the recovered events harbored a single copy of the vector. When applied later in the process (shoot initiation or elongation), the heat shock treatment led to about 30-40% of the events having a single copy of the transforming vector. If heat shock was not applied when employing the heat shock vector, far fewer events were recovered than when the heat shock treatment was applied. This is most likely due to the presence of the ipt gene blocking regeneration. About 55% of the events from transformation with the EF1A excision vector were single copy. Transformation rates when using a vector without an excision mechanism were low, as was the proportion of single copy events. The results indicate that excision results in high proportions of single copy events.

1. A DNA construct for the production of single copy transformants in transgenic plants, comprising:
a) a sequence comprising a non-inducible promoter operably linked to (i) a nucleotide encoding a recombinase and (ii) a 3′ regulatory element, wherein at least the nucleotide encoding the recombinase is flanked by at least two parallel orientation recombinase target sites; and
b) a polynucleotide encoding at least one polypeptide or at least one polyribonucleotide upstream or downstream of the sequence of a) and operably linked to a second promoter operable in a plant cell.

2. The construct of claim 1, wherein the non-inducible promoter is flanked by the at least two parallel orientation recombinase target sites.

3. The construct of claim 2, wherein the 3′ regulatory element is flanked by the at least two parallel orientation recombinase target sites.

4. The construct of claim 1, wherein the construct comprises a T-DNA construct.

5. The construct of claim 4, further comprising a synthetic T-DNA transmission enhancer.

6. The construct of claim 5, wherein the synthetic T-DNA transmission enhancer is an overdrive sequence.

7. The construct of claim 6, wherein the T-DNA construct comprises a left border sequence and a right border sequence, and wherein the synthetic T-DNA transmission enhancer is upstream of the right border sequence.

8. The construct of claim 1, wherein the non-inducible promoter is a constitutive promoter, a tissue-specific promoter or an organ-specific promoter.

9. (canceled)

10. The construct of claim 1, wherein the recombinase is a cre recombinase, an invertase, an integrase, a resolvase or a combination thereof.

11. (canceled)

12. The construct of claim 1, wherein the polynucleotide encodes a polyribonucleotide, and wherein the polyribonucleotide is a promoter hairpin, a microRNA or a non-coding RNA.

13. The construct of claim 1, wherein the polynucleotide encodes a polypeptide, and wherein the polypeptide enhances insect resistance, drought tolerance and nitrogen use efficiency.

14. The construct of claim 1, wherein the polynucleotide encodes a selectable marker and a second polypeptide.

15. The construct of any one of claims 1-3, wherein the at least two parallel orientation recombinase target sites comprise RS, gix, lox, FRT, rox, an integrase, an invertase, a resolvase, or a chimeric recombinase.

16. The construct of claim 15, wherein the at least two parallel orientation recombinase targets sites are lox sites.

17. The construct of claim 1, wherein the polynucleotide of part (b) is upstream of the at least two parallel orientation recombination target sites.

18. A transgenic plant or plant part thereof, comprising the construct of claim 1.

19. The plant or plant part of claim 18, wherein the plant or plant part is a monocot or a dicot.

20. (canceled)

21. The plant or plant part of claim 19, wherein the monocot plant or plant part is maize, sorghum, rice, wheat, sugarcane, oat, rye, triticale, or millet.

22. The plant or plant part of claim 19, wherein the dicot plant or plant part is soybean, alfalfa, canola, cotton, or sunflower.

23. A method for increasing the single copy transformation ratio comprising introducing the construct of claim 1 into a plurality of plant cells to produce a population of transgenic plants, wherein the recombinase is expressed in the plant cells and wherein the population of transgenic plants comprise an increased single copy transformation ratio compared with a single copy transformation ratio of a population of transgenic control plants not containing the construct.

24. The method of claim 23, wherein the population of transgenic plants further comprises an increased number of plants not expressing a backbone of the DNA construct compared with the control plants.

25. The construct of claim 1, wherein the 3′ regulatory element is flanked by the at least two parallel orientation recombinase target sites.
